(19) United States
(12) Patent Application Publication
(10) Pub. No.: US 2009/0209950 A1
(43) Pub. Date: Aug. 20, 2009

(54) ELECTROPHYSIOLOGY CATHETER SYSTEM

(75) Inventor: Niel F. STARKSEN, Los Altos, CA (US)

Correspondence Address: MORRISON & FOERSTER LLP, 755 PAGE MILL RD, PALO ALTO, CA, 94304-1018, US

(73) Assignee: Guided Delivery Systems Inc., Santa Clara, CA (US)

(21) Appl. No.: 12/390,326

(22) Filed: Feb. 20, 2009

Related U.S. Application Data
(60) Provisional application No. 61/030,146, filed on Feb. 20, 2008.

(51) Int. Cl. A61B 18/18; A61B 18/02; A61N 7/00; A61B 18/14; A61B 5/04

(52) U.S. Cl. 606/21; 606/33; 601/2; 606/41; 600/375

(57) ABSTRACT

Described herein are devices and methods for treating tissue, comprising a catheter with a plurality of access sites and a plurality of sensors associated with the access sites. The catheter may be positioned along a tissue surface and the sensors may be used to identify a target site along the tissue surface using the plurality of sensors. Analysis of the tissue surface by the sensors is performed without requiring repositioning of the catheter. In some examples, the access sites of the catheter are side openings along a length of the catheter and the plurality of sensors are electrodes configured to measure electrophysiology parameters. In these examples, the catheter may comprise an internal lumen which permits a treatment device, such as an ablation catheter, to be slidably positioned at the desired target site without requiring displacement of the catheter. In other examples, the catheter may comprise a plurality of fixed ablation elements associated with the plurality of access sites.

### CROSS-REFERENCE TO RELATED APPLICATIONS

The present application claims priority under 35 U.S.C. 119(e) to U.S. Provisional Application No. 61/030,146, filed on Feb. 20, 2008, which is hereby incorporated by reference in its entirety.

### BACKGROUND OF THE INVENTION

Blood returning to the heart from the peripheral circulation and the lungs generally flows into the atrial chambers of the heart and then to the ventricular chambers, which pump the blood back out of the heart. During ventricular contraction, the atrio-ventricular valves between the atria and ventricles, i.e. the tricuspid and mitral valves, close to prevent backflow or regurgitation of blood from the ventricles back to the atria. The closure of these valves, along with the aortic and pulmonary valves, maintains the unidirectional flow of blood through the cardiovascular system. Disease of the valvular apparatus can result in valve dysfunction, where some fraction of the ventricular blood regurgitates back into the atrial chambers.

Traditional treatment of heart valve stenosis or regurgitation, such as mitral or tricuspid regurgitation, involves an open-heart surgical procedure to replace or repair the valve. Current accepted treatments of the mitral and tricuspid valves include: valvuloplasty, in which the affected leaflets are remodeled to perform normally; repair of the chordae tendineae and/or papillary muscle attachments; and surgical insertion of an “annuloplasty” ring, which requires suturing a flexible support ring over the annulus to constrict the radial dimension. Other surgical techniques to treat heart valve dysfunction involve fastening (or stapling) the valve leaflets to each other or to other regions of the valve annulus to improve valve function (see, e.g., U.S. Pat. No. 6,575,971).

### BRIEF SUMMARY OF THE INVENTION

Described herein are devices and methods for treating tissue, comprising a catheter with a plurality of access sites and a plurality of sensors associated with the access sites. The catheter may be positioned along a tissue surface and the sensors may be used to identify a target site along the tissue surface using the plurality of sensors. Analysis of the tissue surface by the sensors is performed without requiring repositioning of the catheter. In some examples, the access sites of the catheter are side openings along a length of the catheter and the plurality of sensors are electrodes configured to measure electrophysiology parameters. In these examples, the catheter may comprise an internal lumen which permits a treatment device, such as an ablation catheter, to be slidably positioned at the desired target site without requiring displacement of the catheter. In other examples, the catheter may comprise a plurality of fixed ablation elements associated with the plurality of access sites.

Also described herein are devices and methods for delivering implants comprising anchors that are secured to tissue. The anchors may be deployed at one or more target sites using an anchor delivery system that includes a tracking assembly. The tracking assembly may be used to identify the location of the anchor delivery system, and to reposition the delivery system if desired. In some embodiments, the tracking assembly includes a signal receiver located on a catheter for tracking signals transmitted or conducted from other known locations internal or external to the body, which are used to determine catheter position. In still other embodiments, a catheter of the anchor delivery system includes one or more components which may be tracked by external sensors, such as a magnet or a signal transmitter. The tracking assembly may be used to generate a model or a map of the body structures containing the target sites and may be correlated to CT or MRI images to provide an alternate process for determining the position of the anchor delivery system.

In certain embodiments, a model or map generated from the tracking assembly may provide additional non-structural information relating to the surrounding tissue or body structures. In some embodiments, for example, impedance or membrane potential mapping may be used to distinguish infarcted myocardium from viable myocardium, myocardial tissue and from annular tissue, or identify cardiac conduction pathways. Localized impedance or membrane potential information of the heart may be used to identify preferred anchor deployment sites, or affect the decision to apply energy or cryotherapy to the target site. Thus, in further embodiments, the anchor delivery system may optionally include an energy-delivery or cryotherapy assembly used in combination with anchor deployment to augment tissue remodeling.

In some embodiments, the tracking assembly may be used in to reduce the need for serial fluoroscopy or CT imaging. These modalities are commonly used during lengthy or complex procedures to confirm the location of the implants or delivery devices, but they expose patients to progressive amounts of ionizing radiation and contrast dye. Furthermore, the model or map generated by the tracking assembly may be used to facilitate the guidance of the anchor delivery system to the desired target sites using magnetic or robotic remote control systems.

In one embodiment, a tissue remodeling system for use in a patient is provided, comprising an anchor delivery catheter comprising a through lumen and a first delivery aperture configured to releasably retain a biased anchor slidably coupled to a tether, a tracking system configured for insertion into a body of a patient and comprising at least one electrode configured to acquire electrical information. In some embodiments, the electrical information may be tissue impedance information or membrane voltage information. At least two surface electrodes may be located about the first delivery aperture. The tissue remodeling system may optionally further comprise a tunnel catheter, wherein the tunnel catheter comprises a catheter lumen with at least one anchor aperture. In some embodiments, at least a portion of the tracking system may be embedded in a wall of the anchor delivery catheter or the tunnel catheter. In some embodiments, the tracking system may further comprise an electrophysiology signal processor configured to receive a signal from the at least one electrode. The tunnel catheter may comprise at least seven or at least eight longitudinally spaced anchor apertures. At least one electrode may be located between each adjacent pair of longitudinally spaced anchor apertures of the tunnel catheter. In some embodiments, the surface electrodes of the tracking system are at least double in number with respect to the number of anchor apertures of the tunnel catheter. The tracking system may further comprise a catheter-embedded antenna assembly and/or a magnetic navigation element. The magnetic navigation element may be located at a distal portion of the delivery catheter or at a distal portion of a guidewire. The tissue remodeling system may also further comprise an energy-delivery assembly. The energy-delivery assembly may be integral with or separate from the anchor delivery catheter.

In another embodiment, a method for securing an anchor to a body structure is provided, comprising providing a first anchor, positioning the first anchor at a first anchor deployment site, assessing a physiologic property of the first anchor deployment site, and deploying the first anchor at the first anchor deployment site. Furthermore, the method may optionally comprise changing the first anchor deployment site based upon the physiologic property, which may include reassessing the physiologic property of the first anchor deployment site after changing the first anchor deployment site. The physiologic property may be an electrical property, which may be a membrane voltage or an impedance. The method may also further comprise positioning a second anchor at a second anchor deployment site, assessing a physiologic property of the second anchor deployment site, and deploying the second anchor at the second anchor site. The method may further comprise retaining a tether coupled to the first anchor and the second anchor after deploying the first anchor and second anchor. The method may also further comprise changing a tissue structure at the first anchor deployment site. In some embodiments, the method may further comprise deploying the first anchor through a first opening of the catheter, deploying the second anchor through a second opening of the catheter, retaining the first coupling portion of the implant in the catheter, wherein the first coupling portion is located between two anchors secured to the body structure, and releasing the first coupling portion of the implant from the catheter after securing the first anchor and the second anchor to body tissue. In some embodiments, releasing the first coupling portion of the implant from the catheter comprises disengaging a wall section of the catheter, and the method may further comprise positioning the catheter in a subvalvular space of a ventricle. Sometimes, changing the tissue structure at the first anchor deployment site comprises causing protein denaturation at the first anchor deployment site, and other times comprises causing at least some tissue ablation at the first anchor deployment site. The method may further comprise cinching the first anchor and the second anchor closer together, and optionally reassessing the physiologic properties of the first and second anchor deployment sites after cinching. Sometimes, the method may further comprise adjusting the cinching of the first anchor and the second anchor based upon reassessing the physiologic properties of the first and second anchor deployment sites and securing the configuration of the cinched first anchor and second anchor. Securing the cinched first anchor and second anchor may occur after reassessing the physiologic properties of the first and second anchor deployment sites. The method may also further comprise assessing a physiologic property of a region located between the first and second anchor deployment sites.

In other embodiments, a method for assessing body tissue is provided, comprising providing an image of a body structure constructed from localized body structure information, positioning an anchor delivery system about the body structure, wherein the anchor delivery system comprises a sensor and an anchor coupled to a tether, taking a localized information reading using the sensor of the anchor delivery system, comparing the localized information reading to the image of the body structure, and deploying the anchor at a target site of the body structure. In some embodiments, the method may further comprise repositioning the anchor delivery system based upon comparing the localized information reading to the image of the body structure. The image of the body structure may be a three-dimensional image, and the localized tissue information may be electrical-based tissue information, such as membrane potential data or impedance data, or may be mechanical tissue information, such as tissue compliance data. The tissue compliance data may be generated using a catheter-based pressure sensor. The method may also further comprise determining an anchor delivery system location.

In another embodiment, a method for treating body tissue is provided, comprising accessing a plurality of cardiac target sites in a patient using a tubular body, deploying a plurality of biased anchors at the plurality of cardiac target sites using the tubular body, wherein the plurality of biased anchors are coupled to a tether member, delivering energy to at least one of the plurality of cardiac target sites using the tubular body in an amount sufficient to at least denature some protein at the at least one of the plurality of cardiac target sites, and withdrawing the tubular body after deploying the plurality of biased anchors and after delivering energy to at least one of the plurality of cardiac target sites.

In another embodiment, a device for assessing tissue is provided, comprising an elongate outer body, a plurality of longitudinally arranged sensor structures associated with a plurality of longitudinally arranged access regions of the elongate body, and wherein each sensor structure comprises a lead wire with a distal end coupled to a sensor structure and a proximal end located about a proximal portion of the elongate outer body. In some examples, the sensor structure may be an electrode structure. The plurality of longitudinally arranged access regions may comprise a plurality of longitudinally arranged access openings. The device may also further comprise a movable inner member within the elongate outer body and may be configured to be selectively positioned at each access region. The movable inner member may comprise an ablation assembly, a tissue injection assembly, a sensor assembly, and/or an anchor delivery assembly.

In another embodiment, a method for evaluating a patient for a cardiac abnormality is provided, comprising positioning a catheter along a portion of an cardiac surface, wherein the catheter comprises a plurality of longitudinally arranged electrodes and at least two side openings, assessing the physiological activity at a plurality of cardiac sites along the portion of the cardic surface without requiring and/or actually repositioning of the catheter, selecting an target site based upon the physiological activity of the plurality of cardiac sites, positioning an active element at the target site, and acting on the target site using the active element and at least one side opening of the catheter. The cardiac surface may be an endocardial surface and wherein the plurality of cardiac sites may be endocardial sites. In some examples, acting on the target site may comprise ablating the target site using an active element that comprises an ablation element. In some examples, assessing the physiological activity at the plurality of cardiac sites may comprise assessing the physiological activity of at least two cardiac sites simultaneously. In some specific examples, the portion of the endocardial surface comprises annular tissue associated with the mitral valve. In further examples, the annular tissue may be subvalvular annular tissue. The method may also further comprise contacting the ablation element to the target site through a side opening, and the ablation element may be selected from a group consisting of radiofrequency ablation element, a cryoablation element and a high intensity focused ultrasound element.

In still another embodiment, a method for evaluating a patient with an arrhythmia is provided, comprising positioning a catheter along a portion of an endocardial surface, wherein the catheter comprises a plurality of longitudinally arranged electrodes and at least one ablation opening, assessing the physiological activity at a plurality of endocardial sites along the portion of the endocardial surface without requiring repositioning of the catheter, selecting an ablation site based upon the physiological activity of the plurality of endocardial sites, positioning an ablation element at the ablation site, and ablating the ablation site using the ablation element and an ablation opening of the catheter. The portion of the endocardial surface may comprise annular tissue associated with the mitral valve, and the annular tissue may be the subvalvular annular tissue. In some embodiments, the method may further comprise contacting the ablation element to the ablation site using the ablation opening. The ablation element may be selected from a group consisting of radiofrequency ablation element, a cryoablation element and a high intensity focused ultrasound element. Also, in some embodiments, positioning the ablation element at the ablation site may be performed without moving the catheter.

### BRIEF DESCRIPTION OF THE DRAWINGS

The structure and method of using the invention will be better understood with the following detailed description of embodiments of the invention, along with the accompanying illustrations, in which:

FIG. 1 is a cross-sectional view of a heart with a guide catheter device advanced through the aorta into the left ventricle;

FIG. 2 is a flowchart representation of a method for delivering at least two anchors into a subvalvular region;

FIGS. 3A to 3D schematically depict a method for delivering multiple tissue anchors using a guide tunnel having multiple tissue anchor openings;

FIG. 4 depicts a transseptal approach to the left ventricle;

FIG. 5 depicts a transapical approach to the left ventricle;

FIGS. 6A and 6B are schematic views of the heart illustrating various dimensions of a heart chamber;

FIG. 7 depicts the use of a multi-opening guide tunnel along a longitudinal portion of the left ventricle;

FIG. 8 is a perspective view of a distal portion of one embodiment of an anchor delivery catheter;

FIGS. 9A and 9B are perspective views of a distal portion of another embodiment of an anchor delivery catheter;

FIG. 10A is a perspective view of another embodiment of a delivery catheter, FIG. 10B is a frontal view of the delivery catheter of FIG. 10A, and FIGS. 10C and 10D are side and bottom views, respectively, of a portion of the delivery catheter of FIG. 10A;

FIG. 11A depicts one embodiment of a multi-opening guide tunnel; FIG. 11B depicts the multi-opening guide tunnel of FIG. 11A with its latches unlocked and separated from the body of the guide tunnel; FIG. 11C illustrates one embodiment of an inner guide tunnel usable with the multi-opening guide tunnel of FIG. 11A; FIGS. 11D and 11E are schematic cross-sectional views of the multi-opening guide tunnel at various locations; FIG. 11F is a schematic illustration of a locking element;

FIGS. 12A to 12H are various perspective views of one embodiment of a multi-opening guide tunnel;

FIG. 13 schematically depicts one embodiment of a tracking assembly;

FIG. 14 schematically depicts another embodiment of a tracking assembly;

FIGS. 15A and 15B are schematic cross-sectional views of a mitral subvalvular region depicting non-contact and contact positions of a guide catheter, respectively;

FIGS. 16A through 16D are schematic cross-sectional views depicting rotational reorientation of a delivery catheter and the deployment of an anchor about the annular tissue;

FIGS. 17A and 17B depict one embodiment of a multi-electrode mapping catheter;

FIGS. 18A and 18B depict another embodiment of a multi-electrode mapping catheter;

FIGS. 19A and 19B depict another embodiment of a multi-electrode mapping catheter;

FIG. 20 depicts an embodiment of a guide tunnel with longitudinal electrodes and tip electrodes;

FIG. 21 depicts an embodiment of a delivery catheter comprising tip electrodes;

FIG. 22 depicts an embodiment of a guide tunnel with a magnetic guidance element;

FIG. 23 depicts a variant of the catheter in FIG. 8, further comprising a plurality of electrodes;

FIGS. 24A through 24C are side, front and bottom elevational views, respectively, of a mapping delivery catheter;

FIGS. 25A through 25C are side, front and bottom elevational views, respectively, of another mapping delivery catheter;

FIGS. 26A through 26C are side, front and bottom elevational views, respectively, of another mapping delivery catheter;

FIG. 27 depicts one embodiment of a multi-aperture catheter with inter-aperture electrodes;

FIG. 28 depicts another embodiment of a multi-aperture catheter with inter-aperture electrodes;

FIGS. 29A and 29B are side and top elevational views of a mapping guide tunnel with an ablation catheter, respectively; FIGS. 29C and 29D are top and cross-sectional views, respectively, of the ablation catheter in FIGS. 29A and 29B;

FIG. 30 depicts a combination mapping and ablation catheter;

FIGS. 31A and 31B are top and side elevational views, respectively, of a mapping guide tunnel and ablation catheter;

FIG. 32 depicts an external ECG tracing of a patient with intracardiac electrograms of various endocardial sites;

FIG. 33 depicts an external ECG tracing intracardiac electrograms of various endocardial sites in a patient with a bypass tract;

FIGS. 34A and 34B depict a cardiac rhythm management anchor-lead in a delivery and deployed configuration, respectively; FIGS. 34C and 34D are perpendicular and planar axial cross-sectional views of the coupling of FIGS. 34A and 34B;

FIG. 35 is a cross-sectional view of another embodiment of an anchor-lead coupling;

FIG. 36 is a cross-sectional view of another embodiment of an anchor-lead coupling; and

FIGS. 37A and 37B illustrate another embodiment of a cardiac rhythm management anchor lead.

### DETAILED DESCRIPTION

Although a number of surgically implanted ventricular devices and procedures, such as the implantation of an annuloplasty ring or edge-to-edge leaflet repair, are available for treating valvular dysfunction, each procedure presents its own set of risks to the patient or technical challenges to the physician.

The devices, systems and methods disclosed herein may be generally used to reshape atrio-ventricular valves or myocardium. The implantation procedures are preferably transvascular, minimally invasive or other “less invasive” surgical procedures, but can also be performed with open or limited access surgical procedures. When used for the treatment of cardiac valve dysfunction, the methods generally involve positioning one or more anchor delivery devices at a target site, delivering slidably coupled anchors from one or more delivery devices, and drawing the anchors together to tighten the annular tissue. The delivery devices may include an elongate catheter with a housing at or near its distal end for releasably housing one or more anchors, as well as guide devices for facilitating advancement and/or positioning of the anchor delivery device(s). The devices may be positioned such that the housing abuts or is close to valve annular tissue, such as the region within the upper left ventricle bound by the left ventricular wall, a mitral valve leaflet and chordae tendineae. Self-securing anchors having any of a number of different configurations may be used in some embodiments.

### I. Annular Tissue Remodeling

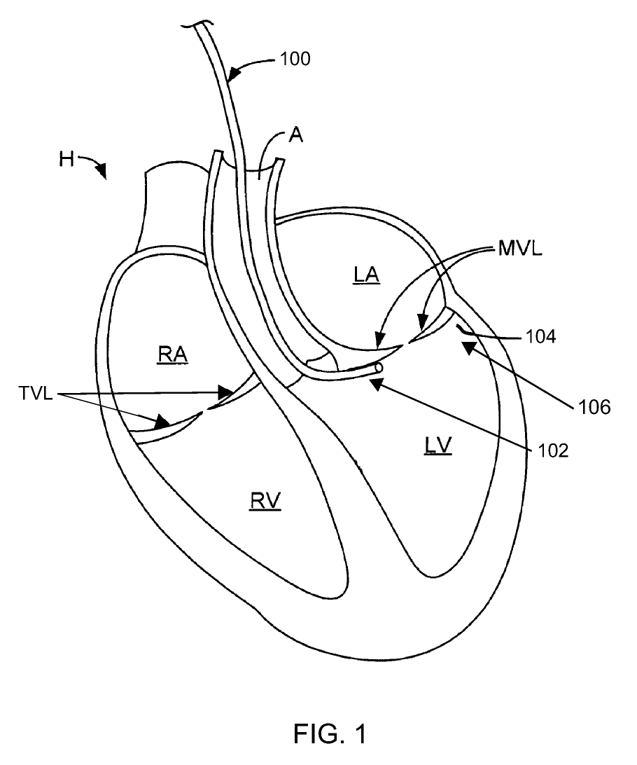

In FIG. 1, a cross-sectional depiction of a heart H is shown with a guide catheter 100 advanced in a retrograde direction through the aorta A and into the left ventricle LV. Retrograde, as used herein, generally refers to a direction opposite the expected flow of blood. In a preferred embodiment, this access route is used to reach the subvalvular space 106. Guide catheter 100 is generally a flexible elongate catheter which may have one or more curves or bends toward its distal end to facilitate placement of the distal end 102 of the catheter 100 at the desired location. The subvalvular space, as used herein, generally includes the portion of the ventricular chamber that is bound by the ventricular wall, the atrio-ventricular valve leaflets, and the chordae tendineae, and travels along most or the entire circumference of the valve annulus. The subannular groove region, as used herein, includes the space bordered by the inner surface of the ventricular wall, the inferior surface of valve leaflets, and the third order chordae tendineae connected directly to the ventricular wall VW and the leaflet. The distal end 102 of guide catheter 100 may be configured to be positioned at an opening into the subvalvular space 106 or within the subvalvular space 106, such that subsequent delivery devices may be passed through guide catheter 100 into the subvalvular space 106. Although retrograde aortic access preferably starts from a percutaneous or peripheral access site, in some embodiments, access may be achieved by an incision in the ascending aorta, descending aorta, aortic arch or iliac arteries, following surgical, thorascopic or laparoscopic access to a body cavity.

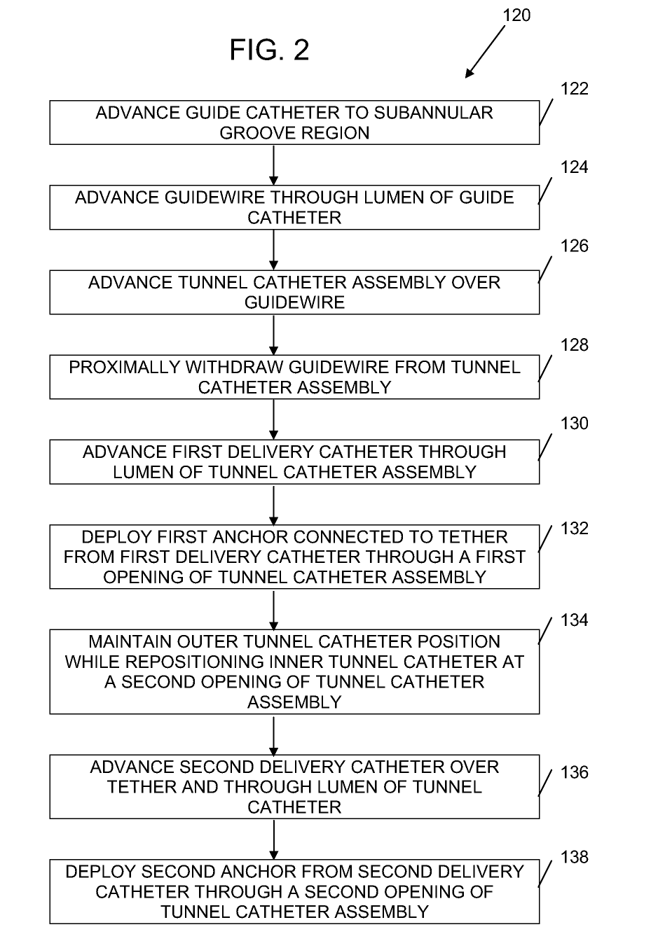

FIG. 2 provides a flowchart depiction of one embodiment comprising a method 120 for deploying at least two anchors of the implant in the region of a heart valve annulus. As shown, the method comprises advancing a guide catheter to the subannular groove region 122, advancing a guidewire through a lumen of the guide catheter 124, advancing a guide tunnel or tunnel catheter over the guidewire 126, and proximally withdrawing the guidewire from the tunnel catheter 128. After the guidewire has been proximally withdrawn, a first delivery catheter may be advanced through the lumen of the tunnel catheter 130 and a first anchor may be deployed into a first region of the heart valve annular tissue 132. In other embodiments, the first delivery catheter may be inserted into guide catheter without the use of a tunnel catheter. The first anchor is typically fixedly attached or otherwise secured to a guide element, such as a tether. In this way, after the first anchor is secured to heart tissue, the guide element will remain attached to the first anchor. While the guide element may be used as a track or monorail for the advancement of additional delivery catheters thereover, the guide element is also a component of the implant that interconnects the multiple anchors. A portion of the guide element facilitates the tightening of the implant and remains in the body with the anchors after the delivery system is removed from the body. Together, the various components used to deliver the anchors, e.g. the guide wire, guide catheter, tunnel catheter and delivery catheter, may be referred to as the anchor delivery system.

After the first anchor has been deployed in the region of the heart valve annular tissue, the first delivery catheter is withdrawn proximally from the tunnel catheter. While maintaining the existing position of the outer catheter of the tunnel catheter about the subannular groove region, the inner catheter of the tunnel catheter is repositioned at a second opening of the outer catheter 134. A second delivery catheter is then advanced over the guide element through the lumen of the tunnel catheter 136. In some embodiments, subsequent delivery of anchors can be achieved by removing and reloading the first delivery catheter. In other embodiments, the delivery catheter is loaded with a plurality of anchors and does not need to be withdrawn from the tunnel catheter to deliver subsequent anchors.

During advancement of the second delivery catheter over the guide element, the guide element may enter the second delivery catheter through an opening or other interface at its distal end, and exit the second delivery catheter through an opening in its side wall that is proximal to its distal end. Alternatively, the guide element may enter the second delivery catheter through an opening at its distal end, and exit the second delivery catheter through an opening at its proximal end, or at any other location proximal to the distal end. After the second delivery catheter has been advanced over the guide element through the lumen of the tunnel catheter, a second anchor is deployed into a second region of the heart valve annular tissue using a second opening of the tunnel catheter 138.

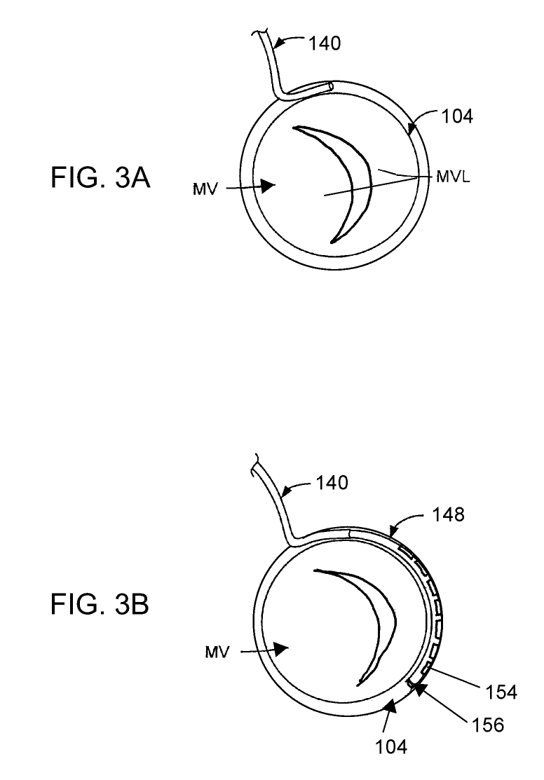

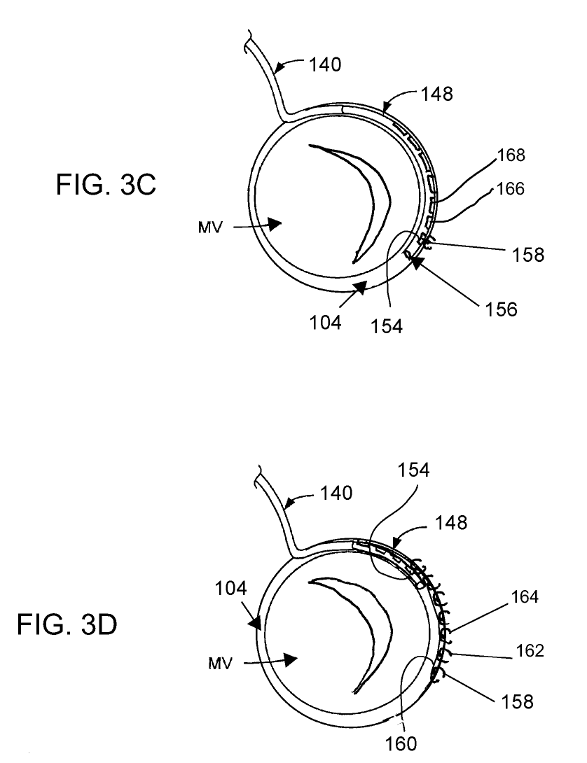

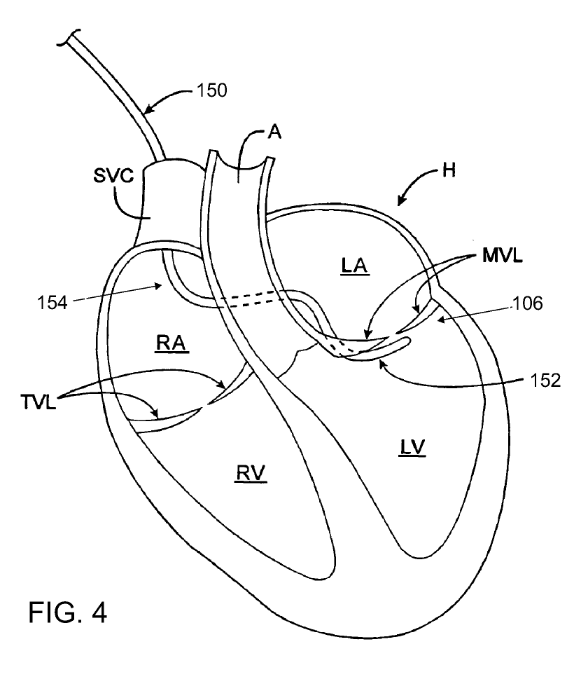

FIGS. 3A to 3D depict one embodiment of the method shown in flowchart form in FIG. 2. In FIGS. 3A to 3D, the mitral valve MV of FIG. 1 is depicted schematically from an inferior perspective looking in a superior direction, but in other embodiments the tricuspid valve, pulmonary valve or aortic valve may be accessed. Referring to FIG. 3A, a guide catheter 140 is advanced to subannular groove region 104 using, for example, any of the access routes (or any other suitable access routes) described herein.

Next, a guide tunnel or tunnel catheter 148 may be advanced through guide catheter 140. Tunnel catheter 148 may be any suitable catheter, and in some instances, it is desirable that the tunnel catheter be pre-shaped or pre-formed at its distal end. In some embodiments, tunnel catheter 148 may have a pre-shaped distal portion that is curved. In this way, the tunnel catheter may more easily conform to the geometry of the atrio-ventricular valve. It should also be understood that any of the catheters or guidewires described here may be pre-shaped or pre-formed to include any number of suitable curves, angles or configurations. Of course, the guidewires and/or catheters described here may also be steerable.

Referring to FIG. 3C, after tunnel catheter 148 has been positioned in the subannular groove region 104, a delivery catheter (not shown) may then be advanced through the lumen of tunnel catheter 148 and toward an opening 154 at or adjacent to the distal tip 156 of tunnel catheter 148. The delivery catheter may remain within tunnel catheter 148, as anchor 158 is deployed through opening 154 to attach to the body tissue. In other embodiments, the delivery catheter may be extended through opening 154 of tunnel catheter 148. Exemplary embodiments of a delivery catheter are depicted and described in greater detail below.

In some embodiments, opening 154 is the distalmost anchor delivery opening of the lumen in tunnel catheter 148, but in some embodiments, one or more openings may have a separate lumen in tunnel catheter 14. Separate lumens permit may permit independent anchor deployment. Furthermore, although FIG. 3B depicts opening 154 as a side opening of tunnel catheter 148, in some embodiments, opening 154 may be located at the distal tip 156.

Anchor 158, shown in FIG. 3C, comprises a self-expanding design. As anchor 158 exits the delivery catheter and tunnel catheter 148, anchor 158 can self-secure into tissue accessible from the subannular groove region 104 or subvalvular space 106. It should be understood that one or more anchors of an implant may be deployed into the annulus directly, while other anchors may be secured to other tissue in the vicinity of the subannular groove region 104 or subvalvular space 106. For example, one or more anchors may be secured to the tissue below the annulus or into the base of the valve leaflet. After anchor 158 has been deployed, the delivery catheter may be proximally withdrawn. A tether 160, attached to anchor 158 and illustrated in FIG. 3D, may be used to facilitate the insertion of additional delivery catheters toward the implantation site.

In this particular embodiment, as demonstrated in FIG. 3D, tunnel catheter 148 is maintained in the same position while additional anchors 162 and 164 are deployed from additional openings 166 and 168 along tunnel catheter 148. In some embodiments, one or more delivery catheters loaded with a single anchor are serially inserted into tunnel catheter 148 using tether 160 to deploy anchors 162 and 164 through openings 166 and 168. In other embodiments, the delivery catheters are configured to hold multiple anchors 158, 162 and 164 and can deliver multiple anchors without requiring withdrawal of the delivery catheter between anchor deployments. Still other multi-anchor delivery catheters are configured to deliver multiple anchors simultaneously through multiple openings of tunnel catheter 148. Anchors 158, 162 and 164 may be deployed from the delivery catheter and tunnel catheter 148 in any suitable fashion, including but not limited to a push-pull wire, using a plunger, or other suitable actuation technique. Similarly, anchors 158, 162 and 164 may be coupled to tether 160 by any suitable attachment method. For example, one or more knots, welded regions, and/or adhesives may be used. Alternate embodiments for anchor deployment and anchor attachments are described in U.S. patent application Ser. No. 11/583,627, which is hereby incorporated by reference in its entirety.

In the embodiments depicted in FIGS. 3A to 3D, before a second delivery catheter is advanced through tunnel catheter 148, tether 160 is threaded into the delivery catheter, and is slidably engaged with a second anchor 162. In some embodiments, second anchor 162 is preloaded into the second delivery catheter before threading to tether 160, while in other embodiments, the second anchor is pre-threaded before being loaded into the second delivery catheter. Any of a number of different methods and threading devices can be used to thread a guide element, such as tether 160, into a delivery catheter, and to engage the guide element with an anchor. Other threading methods and devices are disclosed in U.S. patent application Ser. No. 11/202,474 and U.S. patent application Ser. No. 11/232,190, which are hereby incorporated by reference in their entirety.

With reference to FIG. 3D, after all of anchors 158, 162 and 164 have been deployed into body tissue, tunnel catheter 148 is withdrawn from guide catheter 140. In some embodiments, a termination catheter is inserted through guide catheter 140 over tether 160. The termination catheter is used to facilitate tensioning of tether 160, thereby cinching anchors 158, 162 and 164 together to remodel the annular tissue and to secure the cinched anchors 158, 162 and 164 with a termination member (not shown) that resists tether loosening or slippage. In other embodiments, the termination catheter can secure tether 160 to an anchor or to body tissue without the use of a termination member. Devices and methods for performing termination of cinchable implants are described in U.S. patent Ser. No. 11/232,190, which was previously incorporated by reference, and U.S. patent application Ser. No. 11/270,034 and U.S. patent application Ser. No. 11/255,400, which are hereby incorporated by reference in their entirety.

“Anchors,” for the purposes of this application, are defined to mean any fasteners. Thus, the anchors may comprise C-shaped or semicircular hooks, curved hooks of other shapes, straight hooks, barbed hooks, clips of any kind, T-tags, or any other suitable fastener(s). In one embodiment, anchors may comprise two tips that curve in opposite directions upon deployment, forming two intersecting semi-circles, circles, ovals, helices or the like. In some embodiments, the tips may be sharpened or beveled. In some embodiments, the anchors are self-deforming. By “self-deforming” it is meant that the anchors are biased to change from a first undeployed shape to a second deployed shape upon release of the anchors from a restraint. Such self-deforming anchors may change shape as they are released from a housing or deployed from a lumen or opening to enter annular tissue, and secure themselves to the tissue. Self-deforming anchors may be made of any suitable material such as spring stainless steel, or super-elastic or shape-memory material like nickel-titanium alloy (e.g., NITINOL).

The guide element may be made from any suitable or desirable biocompatible material. The guide element may be braided or not braided, woven or not woven, reinforced or impregnated with additional materials, or may be made of a single material or a combination of materials. For example, the guide element may be made from (1) a suture material (e.g., absorbable suture materials such as polyglycolic acid and polydioxanone, natural fibers such as silk, and artificial fibers such as polypropylene, polyester, polyester impregnated with polytetrafluoroethylene, nylon, etc.), (2) a metal (absorbable or non-absorbable), (3) a metal alloy (e.g., stainless steel), (4) a shape memory material, such as a shape memory alloy (e.g., a nickel titanium alloy), (5) other biocompatible material, or (6) any combination thereof. In some variations, when pulled proximally while restraining the position of the proximal anchor, the guide element may be used to cinch or reduce the circumference of the atrio-ventricular valve annulus or the annular tissue. In certain embodiments, the guide element may be in the form of a wire. The guide element may include multiple layers, and/or may include one or more coatings. For example, the guide element may be in the form of a polymer-coated wire. In certain embodiments, the guide element may consist of a combination of one or more sutures and one or more wires. As an example, the guide element may be formed of a suture that is braided with a wire. In some embodiments, the guide element may be formed of one or more electrode materials. In certain embodiments, the guide element may be formed of one or more materials that provide for the telemetry of information (e.g., regarding the condition of the target site).

In some embodiments, the guide element may include one or more therapeutic agents (e.g., drugs, such as time-release drugs). As an example, the guide element may be partially or entirely coated with one or more therapeutic agents. In certain variations, the guide element may be used to deliver one or more growth factors and/or genetic regenerative factors. In some variations, the guide element may be coated with a material (e.g., a polymer) that encapsulates or controls the release rate one or more therapeutic agents, or in which one or more therapeutic agents are embedded. The therapeutic agents may be used, for example, to treat the target site to which the guide element is fixedly attached or otherwise secured. In certain variations, the guide element may include one or more lumens through which a therapeutic agent can be delivered.

Other embodiments also include treatment of the tricuspid valve annulus, tissue adjacent the tricuspid valve leaflets TVL, or any other cardiac or vascular valve. Thus, although the description herein discloses specific examples of devices and methods for mitral valve repair, the devices and methods may be used in any suitable procedure, both cardiac and non-cardiac. For example, in other embodiments, the mitral valve reshaping devices and procedures may be used with the tricuspid valves also, and certain embodiments may also be adapted for use with the pulmonary and aortic valves. Likewise, the other examples provided below are directed to the left ventricle, but the devices and methods may also be adapted by one of ordinary skill in the art for use in the right ventricle or either atrium. The devices and methods may also be used with the great vessels of the cardiovascular system, for example, to treat aortic root dilatation.

Access to the other chambers of the heart may be performed through percutaneous or venous cut-down access, including but not limited to transjugular, subclavicular and femoral vein access routes. When venous access is established, access to the right atrium RA, the right ventricle RV, the tricuspid valve TV and other right-sided cardiac structures can occur. Furthermore, access to left-sided heart structures, such as the left atrium LA, left ventricle LV, mitral valve and the aortic valve, may be subsequently achieved by performing a transseptal puncture procedure. FIG. 4, with a heart H is shown in cross-section, depicts one embodiment comprising a transseptal puncture procedure. Transseptal puncture is traditionally performed using a Mullins introducer sheath with a Brockenbrough curved needle through the interatrial septum to access the left atrium LA, but any of a variety of other transseptal puncture devices or kits may also be used. After puncturing through the left atrium LA, supravalvular access to the mitral valve is achieved. Antegrade access to the left ventricle LV can also occur by providing through the mitral valve. Similarly, access from the right ventricle RV to the left ventricle LV may be obtained by transseptal puncture of the ventricular septum. In still other embodiments, a catheter device may access the coronary sinus and a valve procedure may be performed directly from the sinus.

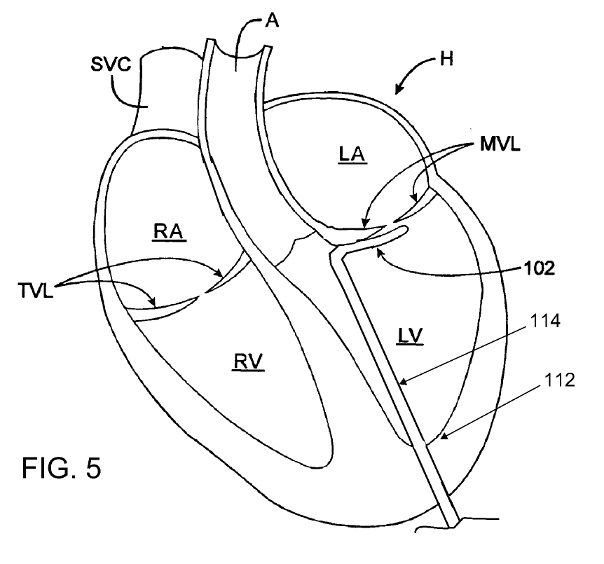

Surgical approaches that may also be used include but are not limited to transcatheter procedures made through surgical incisions in the aorta or myocardium. In one particular embodiment, depicted in FIG. 5, a transapical approach with a surgical delivery device 114 is utilized, to provide a more linear route to the subvalvular space 106. The transapical approach also reduces potential effects of a myocardial incision on cardiac output, as the apical wall 112 typically contributes less mechanical effect on left ventricular ejection fraction compared to other sections of the myocardial wall.

In some embodiments, hybrid access involving a combination of access methods described herein may be used. In one specific example, dual access to a valve may be achieved with a combination of venous and arterial access sites. User manipulation of both ends of a guidewire placed across a valve may improve positioning and control of the catheter and the implants. In other examples of hybrid access, both minimally invasive and surgical access is used to implant one or more cardiac devices.

### II. Ventricular Remodeling

In additional to performing valve annuloplasty, other uses, including cardiac and non-cardiac applications, are contemplated within the scope. In one embodiment, reconfiguration of the subvalvular apparatus with a cinchable implant delivered by an anchor delivery system. For example, a plurality of tethered anchors may be secured to the myocardium adjacent the papillary muscle and then cinched to tension the myocardium and cause repositioning of one or more papillary muscles.

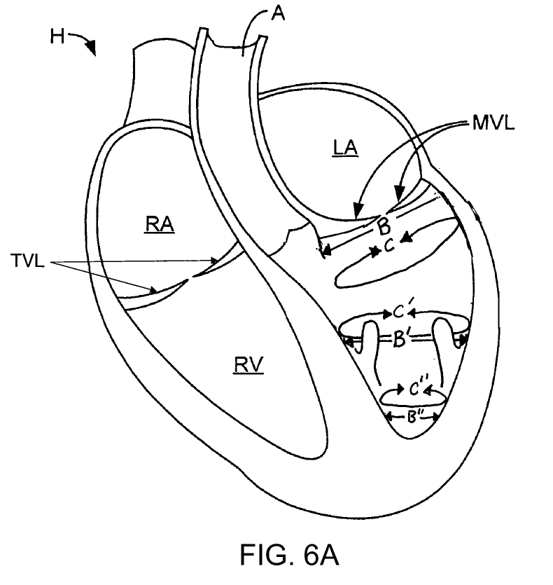

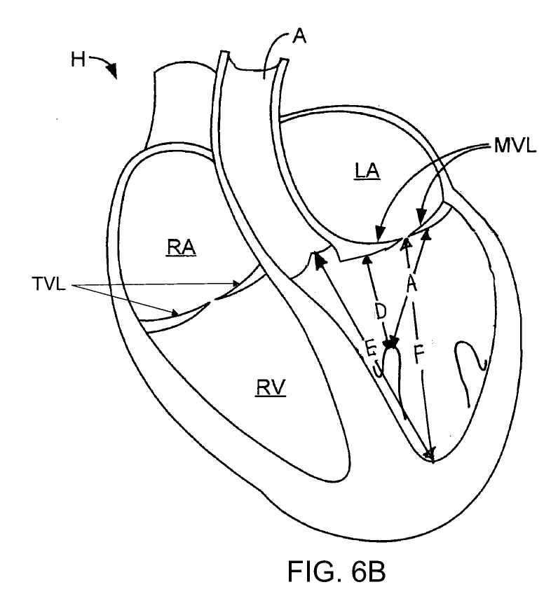

In other embodiments, the reshaping of a heart chamber, such as a ventricle, may be performed along any of a variety of dimensions or vectors. For example, referring to FIG. 6A, in some embodiments, the reshaping of a ventricle or a valve may occur with respect to the diameter B or the circumference C about a valve orifice. In one preferred embodiment, the diameter B and the circumference C with respect to the subannular groove region 104 of a ventricle is reshaped. In addition to the reshaping of to valvular structures, reshaping can also be performed with respect to the non-valvular structures of a heart chamber. For example, one or more of the diameters or circumferences of the ventricle may be reshaped. As shown in FIG. 6A, the diameter B′ and the circumference C′ of the ventricle located generally at or above the papillary muscles may be reshaped. The diameter B″ and circumference C″ of the ventricle at or below the papillary muscles may also be reshaped. The orientation of the diameter and circumference that is reshaped or assessed can vary, but in some embodiments, the diameter or circumference may be in a generally perpendicular orientation with respect to a longitudinal axis of a ventricle. One of skill in the art will understand that the longitudinal axis may be characterized in a number of ways, including but not limited to a longitudinal axis from a valve orifice to an apex of a heart chamber, or from the apex of a heart chamber to a point that generally splits the ventricular volume in half. Similarly, some of the implantation dimensions or vectors may also be oriented with respect to the anterior-posterior axis or the septo-lateral axis of the heart chamber.

Referring to FIG. 6B, in some embodiments, the myocardium along vectors A, D between a papillary muscle and a valve leaflet may be reshaped. Vectors D or A may be between a papillary muscle and its associated valve leaflet, or between a papillary muscle and an unassociated valve leaflet, respectively. Although the vectors A, D depicted in FIG. 6B are shown from the tip of the papillary muscle, these pathways may also be assessed from the base of the papillary muscle. Similarly, myocardial pathways including a valve leaflet may be assessed from the distalmost section, the middle or the base of the valve leaflet. In other embodiments, the reshaping of the heart may occur between the apex of a heart chamber and one or more valves. For example, reshaping may occur along the vector E between the outlet valve and the apex of a heart chamber, and/or along the pathway F between the inlet valve and the apex.

In FIG. 7, for example, a multi-opening guide tunnel 850 with latches 852, described in greater detail below, is used to place a cinchable implant 854 along vector E from FIG. 6B. In some embodiments, one end 856 of the implant 854 is first attached to a less mobile portion of the ventricle chamber, such as the apical region 858 of the left ventricle LV. Once the distal end 856 of the implant 854 is stabilized, guide tunnel 850 can be stabilized using the secured distal end 854 and provide increased stability during the procedure by releasably retaining portions of the tether 860 as the remaining anchors are deployed.

### III. Delivery Catheter

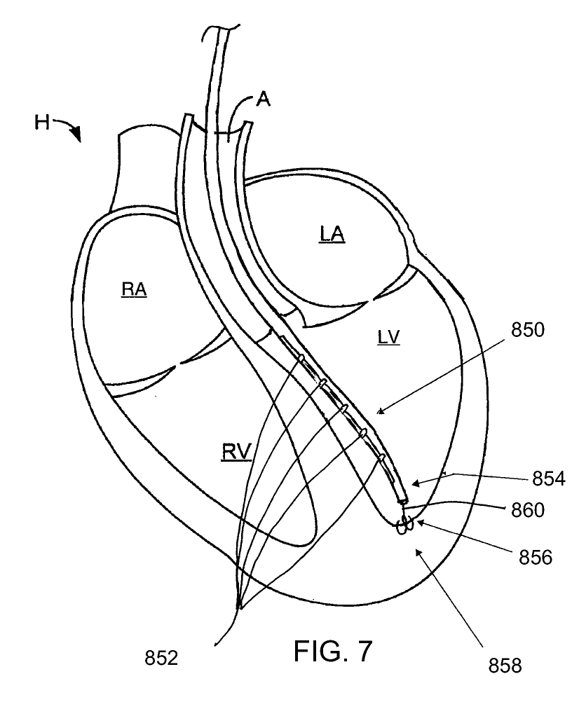

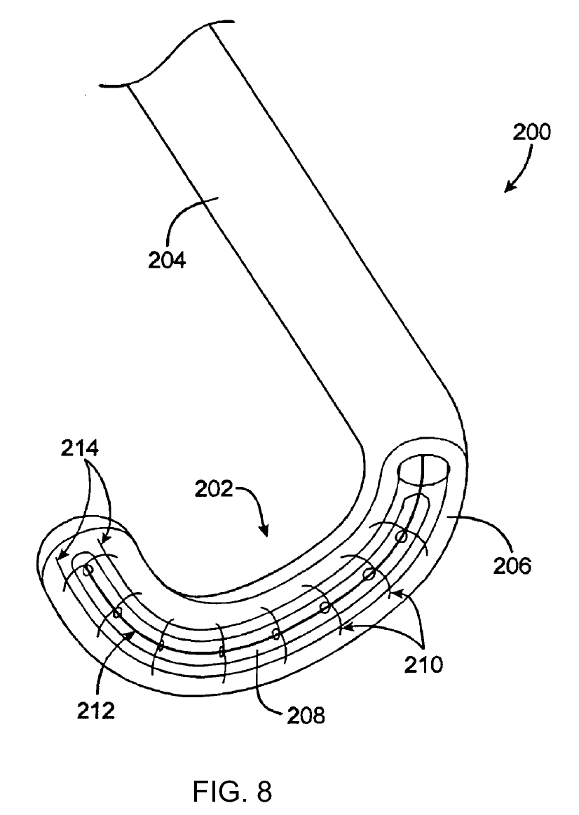

With reference now to FIG. 8, one embodiment comprises an anchor delivery device 200, which suitably includes an elongate shaft 204 having a distal portion 202 configured to deliver a plurality of anchors 210 coupled with a tether 212, and configured for attachment to annular tissue. The tethered anchors 210 are retained by a housing 206 of the distal portion 202, along with one or more anchor retaining mandrels 214. Housing 206 comprises a delivery opening 208 through which anchors 210 are deployed. Embodiments may include one or more of these features, and various parts may be added or eliminated. Some of these variations are described further below, but no specific variation(s) should be construed as limiting.

Housing 206 may be flexible or rigid in some variations. In some embodiments, for example, flexible housing 206 may comprise multiple segments configured such that housing 206 is deformable by tensioning a tensioning member coupled to the segments. In some embodiments, housing 206 is formed from an elastic material having a geometry selected to engage and optionally shape or constrict the annular tissue. For example, the rings may be formed from spring stainless steel, super-elastic shape memory alloys such as nickel-titanium alloys (e.g., Nitinol), or the like. In other embodiments, the housing 206 could be formed from an inflatable or other structure that can be selectively rigidified in situ, such as a gooseneck or lockable element shaft, any of the rigidifying structures described above, or any other rigidifying structure.

In some embodiments, anchors 210 are generally C-shaped or semicircular in their undeployed form, with the ends of the “C” being sufficiently sharp to penetrate tissue. Between the ends of the C-shaped anchor 210, an eyelet may be formed for allowing slidable passage of the tether 212. To maintain the anchors 210 in their C-shaped, undeployed state, anchors 210 may be retained within housing 206 by two mandrels 214, one mandrel 214 retaining each of the two arms of the C-shape of each anchor 210. Mandrels 214 may be retractable within elongate catheter body 204 to release anchors 210 and allow them to change from their undeployed C-shape to a deployed shape. The deployed shape, for example, may approximate a partial or complete circle, or a circle with overlapping ends, the latter appearing similar to a key ring. Such anchors are described further below, but generally may be advantageous in their ability to secure themselves to annular tissue by changing from their undeployed to their deployed shape. In some variations, anchors 210 are also configured to lie flush with a tissue surface after being deployed. By “flush” it is meant that no significant amount of an anchor protrudes from the surface, although some small portion may protrude.

The retaining mandrels 214 may have any suitable cross-sectional shape, cross-sectional area, length and be made of any suitable material, such as stainless steel, titanium, nickel-titanium alloys (e.g., Nitinol), or the like. Some embodiments may not include a mandrel, or may have one mandrel, two mandrels, or more than two mandrels. Mandrels 214 may be configured with indicia, or mechanicals stops or detents, to facilitate a controlled withdrawal of mandrels 214 and release of anchors 210, or to reduce the risk of inadvertent anchor deployment.

In some embodiments, the anchors 210 may be released from mandrels 214 to contact and secure themselves to annular tissue without any further force applied by the delivery device 200. Some embodiments, however, may also include one or more expandable members or force members, which may be expanded or actuated to help drive anchors 210 into tissue. Expandable member(s) and force members may have any suitable size and configuration and may be made of any suitable material(s). Any of a variety of mechanical, pneumatic and hydraulic expandable members known in the art may be included in housing.

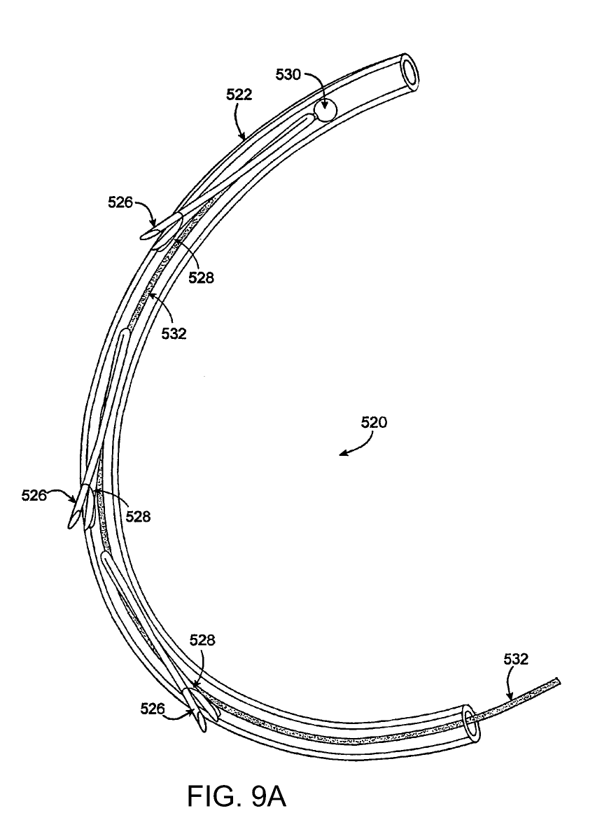

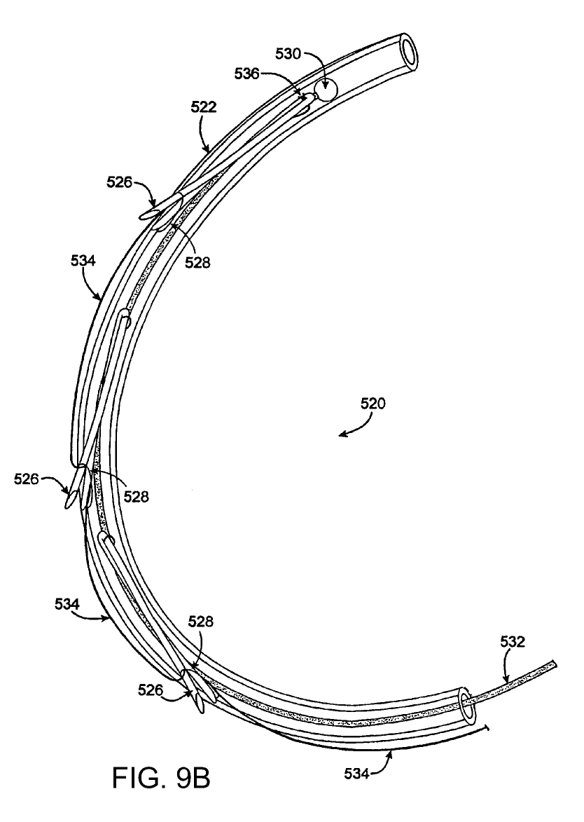

In another embodiment, shown in FIGS. 9A and 9B, a flexible distal portion of an anchor delivery device 520 includes a housing 522 configured to house multiple coupled anchors 526 and an anchor contacting member 530 coupled with a pull cord 532. Housing 522 may also include multiple apertures 528 for allowing egress of anchors 526. For clarity, delivery device 520 is shown without a tether in FIG. 9A, but FIG. 9B shows that a tether 534 may extend through an eyelet, loop or other portion of each anchor 526, and may exit each aperture 528 to allow for release of the plurality of anchors 526. In this particular embodiment, anchors 526 are relatively straight and lie relatively in parallel with the long axis of delivery device 522. Anchor contacting member 530, which may comprise any suitable device, such as a ball, plate, hook, knot, plunger, piston, or the like, generally has an outer diameter that is nearly equal to or slightly less than the inner diameter of housing 522. Contacting member 530 is disposed within the housing, distal to a distal-most anchor 526, and is retracted relative to housing 522 by pulling pull cord 532. When retracted, anchor contacting member 530 contacts and applies force to a distal-most anchor 526 to cause release of that anchor 526 from housing 522 via one of the apertures 528. Contacting member 530 is then pulled farther proximally to contact and apply force to the next anchor 526 to deploy that anchor 526, and so on.

Retracting contacting member 530 to push anchors 526 out of apertures 528 may help cause anchors 526 to secure themselves to the tissue adjacent the apertures 528. Using anchors 526 that are relatively straighter/flatter configuration when undeployed may allow anchors 526 with relatively large deployed sizes to be disposed in (and delivered from) a relatively small housing 522. In one embodiment, for example, anchors 526 that deploy into a shape approximating two intersecting semi-circles, circles, ovals, helices, or the like, and that have a radius of one of the semi-circles of about 3 mm may be disposed within a housing 522 having a diameter of about 6 French (2 mm) and more preferably about 5 French (1.67 mm) or even smaller. Such anchors 526 may measure about 6 mm or more in their widest dimension. In some embodiments, housing 522 may have a diametrical dimension (“d”) and anchor 526 may have a diametrical dimension (“D”) in the deployed state, and the ratio of D to d may be at least about 3.5. In other embodiments, the ratio of D to d may be at least about 4.4, and more preferably at least about 7, and even more preferably at least about 8.8. These are only examples, however, and other larger or smaller anchors 526 may be disposed within a larger or smaller housing 522. The dimensions of an anchor may vary depending on the particular usage. For example, anchors used for ventriculoplasty may permit the use of larger anchors than those used for annuloplasty due to fewer space constraints in the main compartment of the ventricles than in the subvalvular spaces. Furthermore, any convenient number of anchors 526 may be disposed within housing 522. In one variation, for example, housing 522 may hold about 1 to about 20 anchors 526, and more preferably about 3 to about 10 anchors 526. Other variations may hold more anchors 526.

Anchor contacting member 530 and pull cord 532 may have any suitable configuration and may be manufactured from any material or combination of materials. In alternative embodiments, contacting member 530 may be pushed by a pusher member to contact and deploy anchors 526. Alternatively, any of the anchor deployment devices and methods previously described may be used.

Tether 534, as shown in FIG. 9B, may comprise any of the tethers 534 or tether-like devices already described above, or any other suitable device. Tether 534 is generally attached to a distal-most anchor 526 at an attachment point 536. The attachment itself may be achieved via a knot, weld, adhesive, or by any other suitable attachment mechanism. Tether 234 then extends through an eyelet, loop or other similar configuration on each of the anchors 526 so as to be slidably coupled with the anchors 526. In the particular embodiment shown, tether 534 exits each aperture 528, then enters the next-most-proximal aperture, passes slidably through a loop on an anchor 526, and exits the same aperture 528. By entering and exiting each aperture 528, tether 534 allows the plurality of anchors 526 to be deployed into tissue and cinched. Alternate embodiments of housing 522, anchors 526 and tether 534 may also be used. For example, housing 522 may include a longitudinal slit through which tether 534 may pass, thus allowing tether 534 to reside wholly within housing before deployment.

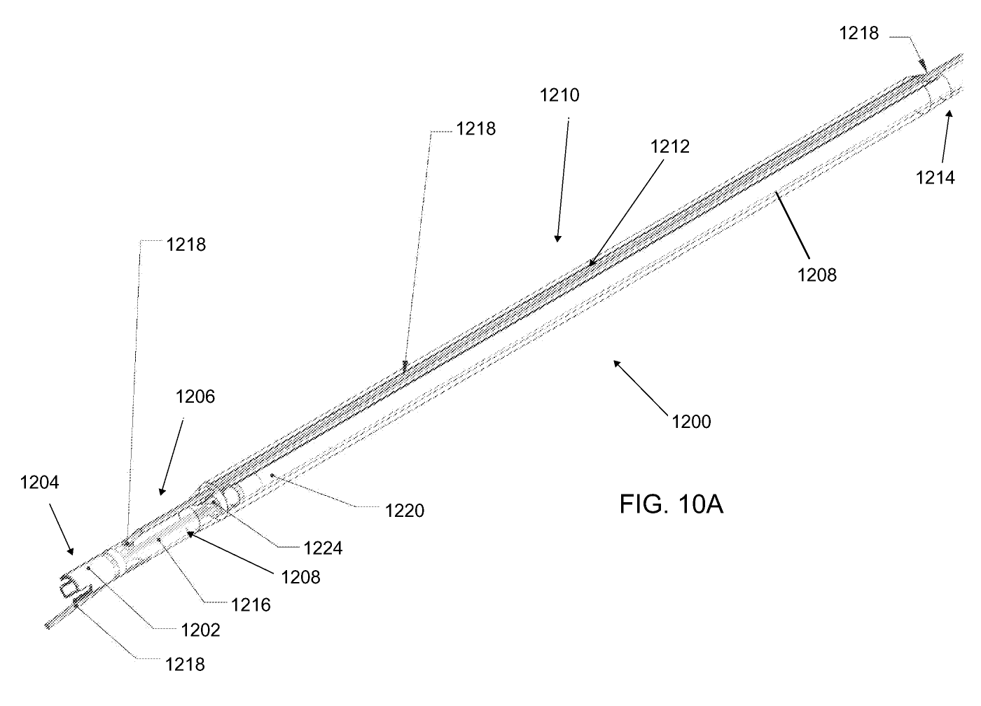

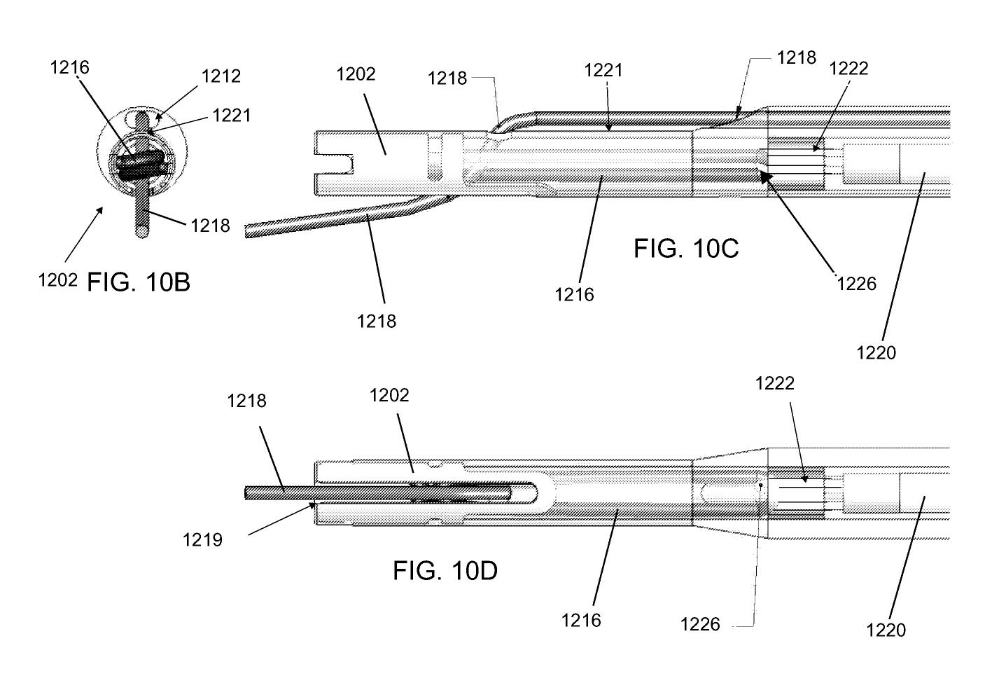

FIGS. 10A to 10D represent various views of one embodiment of a delivery catheter 1200 that can be used to deliver one or more anchors to a target site. As shown in FIG. 10A, delivery catheter 1200 has a distal region 1204 including a tip 1202, an anchor-holding region 1206 including a primary lumen 1208, an intermediate region 1210 including both primary lumen 1208 and a secondary lumen 1212, and a proximal region 1214 including primary lumen 1208. An anchor 1216 is disposed within primary lumen 1208, in the anchor-holding region 1206. While only one anchor is shown in the anchor-holding region of this embodiment, in other embodiments, the delivery catheters may include an anchor-holding region that is adapted to hold multiple anchors. Similarly, while the embodiment shown in FIGS. 10A to 10D depicts anchors adapted to be deployed from distal region 1204 of delivery catheter 1200, it should be understood that the anchors may be deployed from any suitable region of delivery catheter 1200, as desirable. For example, if desirable, the anchor may be delivered out of a side port or hole on the delivery catheter.

As shown in FIGS. 10A to 10D, a tether 1218 may be threaded into a slot 1219 of tip 1202 (shown in FIGS. 10C and 10D), and through an eyelet 1226 of anchor 1216. After extending through eyelet 1226, tether 1218 exits primary lumen 1208, and extends along an exterior surface 1221 of delivery catheter 1200 for the remainder of the length of the anchor-holding region, as shown in FIG. 10C. Tether 1218 then enters secondary lumen 1212, and extends through the length of secondary lumen 1212, exiting secondary lumen 1212 at an end of distal region 1214. An actuator 1220 is slidably disposed within primary lumen 1208, and can be used to push or deploy anchor 1216 out of the primary lumen 1208. Actuator 1220 is in the form of a pushable generally tubular member, although other forms of actuators may be used. For example, in some variations, a solid rod may be used as an actuator, and may be optionally motor-controlled. Once a sufficient distal portion of anchor 1216 has been displaced out of primary lumen 1208, the self-expanding properties of anchor 1216 may cause the biased distal ends to expand outwardly and cause the remainder of anchor 1216 to “spring out” or “shoot out” of distal end 1202 and facilitate tissue piercing by anchor 1216. Eyelet 1226 will also engage tether 1218 as anchor 1216 exists delivery catheter 1200. In other embodiments, actuator 1220 may be spring-loaded or biased to facilitate tissue piercing. Additional embodiments of the delivery catheter are described in U.S. patent application Ser. No. 11/202,474, which was previously incorporated by reference.

Delivery catheter 1200 may optionally comprise a retaining or retrieval member, such as a retrieval suture 1222 that is looped around eyelet 1226 of anchor 1216 and threaded proximally back through delivery catheter 1200. Retrieval suture 1222 is pulled of delivery catheter 1200 by eyelet 1226 when anchor 1216 is deployed. Retrieval suture 1222 may be used to at least partially pull back anchor 1216 into delivery catheter 1200 should anchor 1216 misfire and fail to engage body tissue. If anchor 1216 is successfully deployed, one end of retrieval suture 1222 may be pulled out from eyelet 1226 to release anchor 1216 from retrieval suture 1222.

### IV. Guide Tunnel

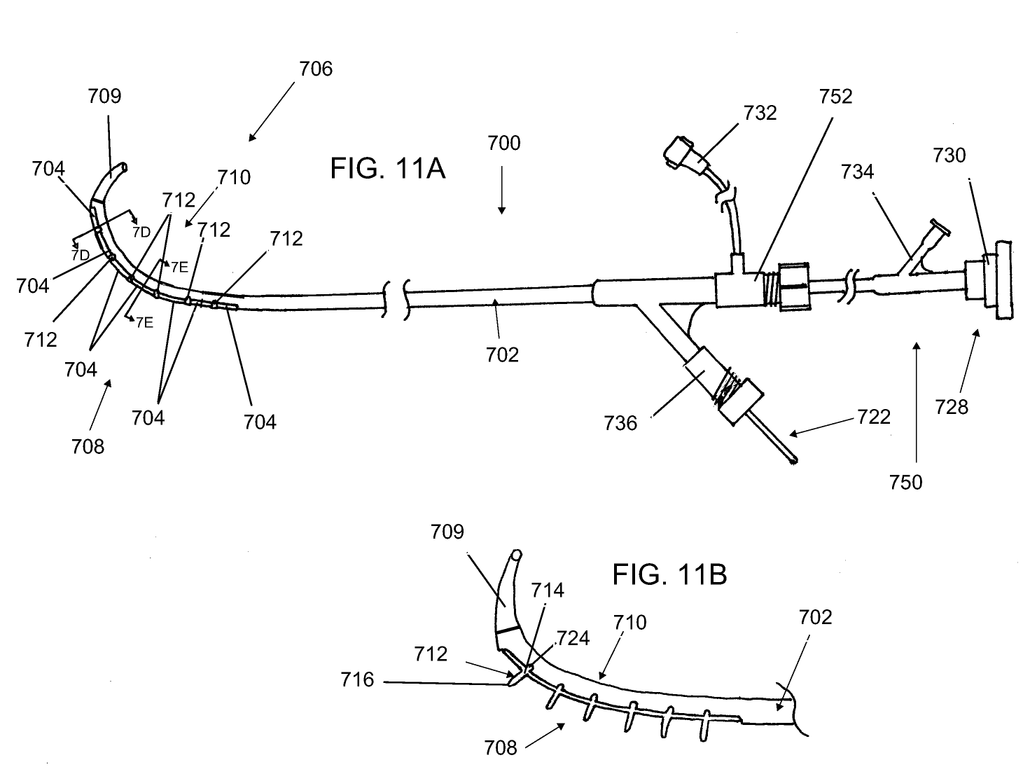

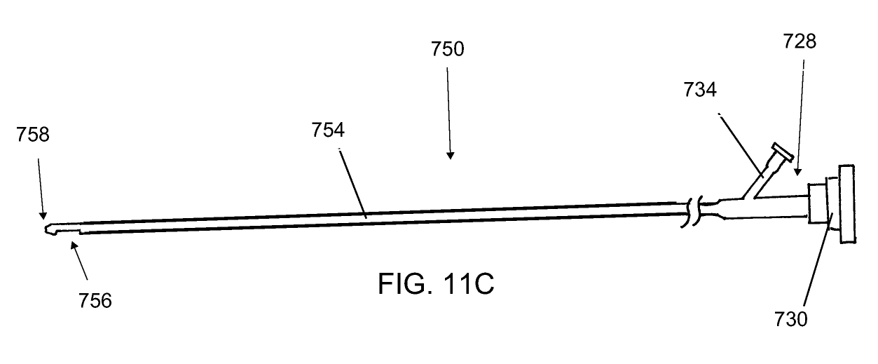

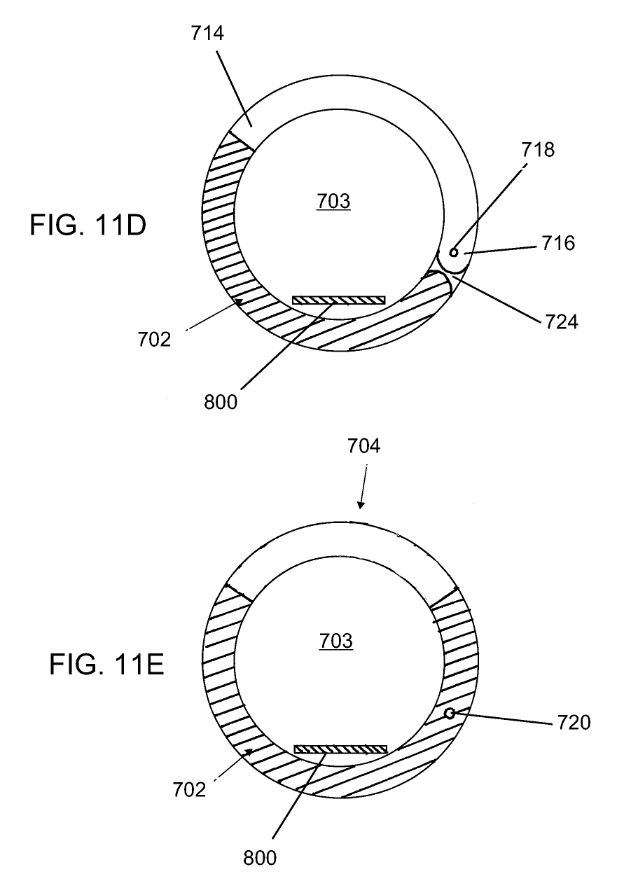

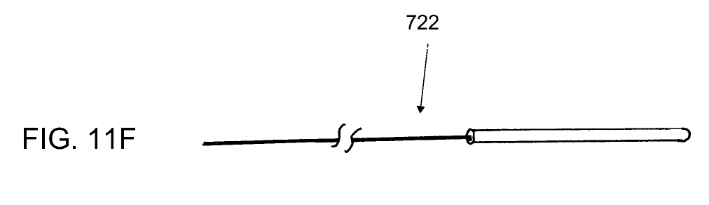

Referring now to FIGS. 11A through 11F, one embodiment of a guide tunnel 700 that may be used as a component of the anchor delivery system comprises a tubular body 702 with a central passageway 703 and multiple anchor openings 704. Central passageway 703, depicted in FIGS. 11D and 11E, permits the insertion of a delivery catheter (or other device) with alignment of one or more retained anchors to one or more of the anchor openings 704 of guide tunnel 700. Typically, anchor openings 704 are grouped in a distal portion 706 of guide tunnel 700, but in other embodiments, anchor openings 704 may be located more proximally. The lengths and configurations of the tubular body 702 and distal portion 706 may vary depending upon a variety of factors, including but not limited to the desired target location, such as the subannular groove region, and the access route, whether it is retrograde or antegrade, or requires a transseptal puncture. In one example, distal portion 706 of guide tunnel 700 comprises a flexible curved configuration. In some embodiments, anchor openings 704 are preferably aligned along the greater curvature 708 of distal portion 706. In other embodiments, anchor openings 704 may be aligned along the superior junction of the curved distal portion. Similarly, guide tunnel 700 may be configured for a cinchable implant inserted via the coronary sinus by aligning anchor openings 704 along the lesser curvature 710 of distal portion 706. Distal portion 706 may optionally comprise an atraumatic tip, such as an inflatable balloon or a tapered tip 709 comprising a material with a low durometer. Guide tunnel 700 may be used in conjunction with a guide catheter to facilitate positioning of a delivery catheter at the desired anchoring sites.

In some embodiments, anchor openings 704 are arranged in a linear configuration along a longitudinal length of guide tunnel 700, while in other embodiments, anchor openings 704 may be offset along the circumference of guide tunnel 700. Although anchor openings 704 are depicted in FIG. 11A through 11E as having uniform dimensions, uniform spacing and angular and linear alignment, these and other features of guide tunnel 700 may be varied as desired. For example, if the cinchable implant comprises anchors of different sizes and anchor spacings, the anchor opening cross-sectional areas and relating spacing may be designed accordingly.

Guide tunnel 700 may be used in beating heart procedures where it is difficult to control the position of the distal end of a delivery catheter with respect to the target tissue. By providing multiple anchor openings 704, once guide tunnel 700 has been positioned at its desired location, its position may be maintained while deploy a plurality of anchors. Instead, a delivery catheter can be manipulated within the non-moving guide tunnel 700 to deploy the anchors through the provided anchor openings 704. Thus, guide tunnel 700 may reduce the risk that, during a procedure involving multiple anchoring sites, repositioning of the delivery catheter to a new target location may dislodge the delivery catheter from a hard-to-reach target site that are easily lost. Guide tunnel 700, however, may still be moved during a procedure if desired. In addition to transluminal procedures, guide tunnel 700 may also be used with open or limited access surgeries. In further embodiments, guide tunnel 700 may be configured with a shorter longitudinal length and/or a more rigid body for some surgical applications.

During the deployment of a cinchable implant, when the anchors have been secured to their target sites, the coupling members or one or more segments of the tether may still be looped within the delivery catheter or guide tunnel 700. This may be beneficial when implanting anchors in unstable body regions such as a beating heart because with each deployment of an anchor, the retention of a tether segment in guide tunnel 700 further secures guide tunnel 700 to the sites where the anchors have been secured. Once all of the anchors have been deployed, however, the retained tether segments may be separated from guide tunnel 700 so that guide tunnel 700 may be withdrawn.

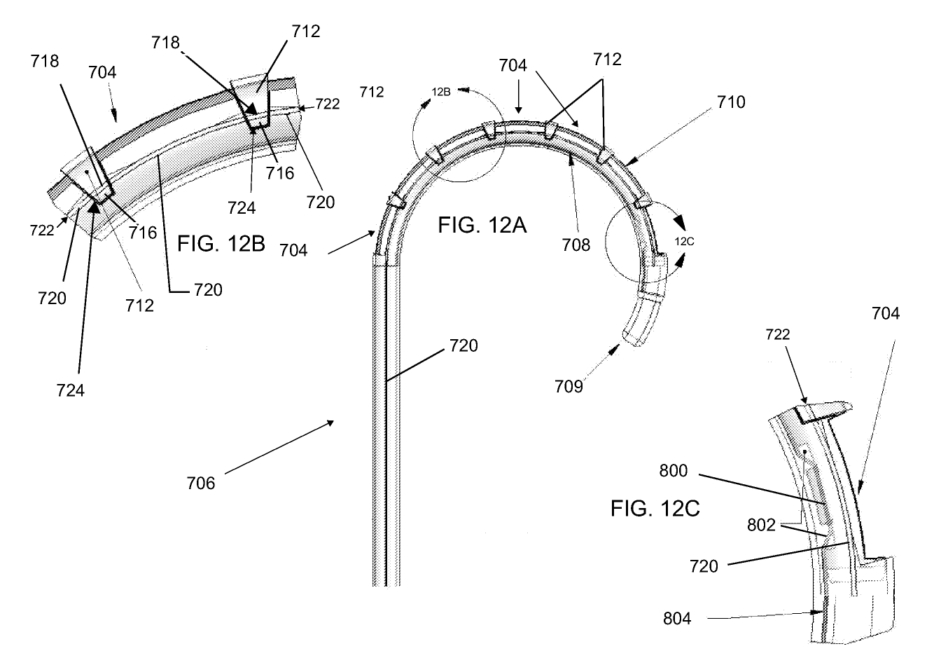

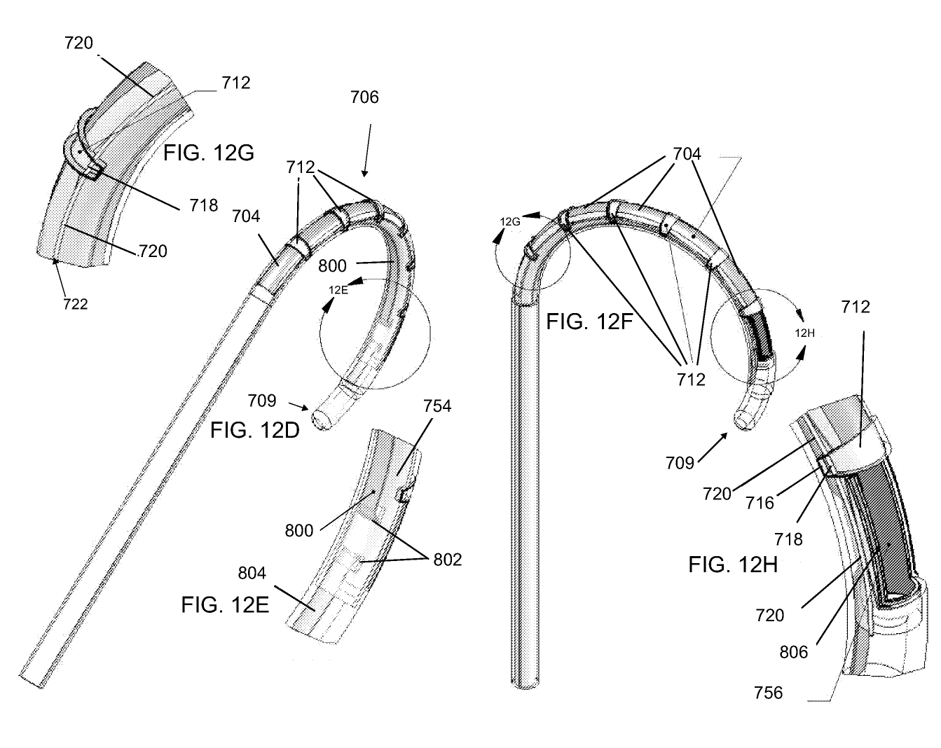

In one embodiment, the retaining structures between anchor openings 704 may be configured to releasably retain the tether or coupling elements between the anchors. In a further embodiment, depicted in greater detail in FIGS. 12A through 12H, the retaining structures comprise wall segments or latch structures 712 located between two adjacent anchor openings 704 of guide tunnel 700. Referring to back to FIG. 11B, which depicts latches 712 of guide tunnel 700 pulled away from tubular body 702, in some embodiments, latch 712 may comprise a base 714 and a free end 716. In some embodiments, latch 712 comprises a material and/or configuration to permit some deformation or displacement of latch 712 and for a tether or coupling member retained between two adjacent anchor openings 704 to pass out of guide tunnel 700.

Referring to FIG. 12B, latch 712 may be configured to permit control of the retention and/or release of the tether between deployed anchors. In some embodiments, latch 712 comprises a lumen 718 that is alignable with complementary segments 720 of a lumen located in the wall of the tubular body 702. The complementary lumen segments 720 may be provided in a notched region 724 which is complementary to free end 716 of latch 712. When aligned, each adjacent lumen 718 and segment of the longitudinal lumen 720 permits the insertion of a locking element 722. Locking element 722 is depicted separately in FIG. 11F. Locking element 722 can form a reversible interference fit the between the lumen 718 of latch 712 and lumen segment 720 of tubular body 702, thereby restricting the passage of a coupling member. When anchors are deployed through anchor openings 704 adjacent to latch 712, the tether will be retained by latch 712.

In some embodiments, locking element 722 may have an elongate configuration and comprise a wire or a plastic. Referring back to the embodiment depicted in FIG. 11A, latch 712 comprise transverse through lumens 718 that complement the lumen segments of the longitudinal lumen 720 of the tubular body 702, but the particular orientations of the lumens or locking elements may vary, depending on the desired orientation of anchor openings 704. Lumen 718 of latch 712 need not be a through lumen or a transversely oriented lumen with respect to the base 714 and free end 716 of latch 712. In some embodiments, latches 712 may comprise radio-opaque material to facilitate the positioning of a delivery catheter with respect to guide tunnel 700. In other embodiments, radio-opaque material may be located in or on tubular body 702 in angular position generally opposite one or more latches 712 or elsewhere.

In some embodiments, latch 712 may not maintain the alignment of lumen 718 with its complementary lumens 720 once locking element 722 is removed. In these embodiments, reinsertion or rethreading of locking element 722 back into lumen 718 may not work in situ. In other embodiments, however, guide tunnel 700 may be constructed such that latch 712 is biased to an alignment position and locking element 722 may be reengaged to one or more lumens 718, 720. To facilitate initial insertion or reinsertion of locking element 722 into lumens 718, 720, lumens 718, 720 may be provided with one or more flanged lumen openings.

In some embodiments, a single locking element 722 is provided and is insertable through all lumens 718 of latch 712 and complementary lumens 720 of tubular body 702, and the aggregate lumen path from lumens 718 and complementary lumens 720 is substantially linear or curvilinear. With these particular embodiments, release of latches 712 with start with the distalmost latch and finish with the most proximal latch. In other embodiments, the lumens and the locking element, such as the locking element 724 shown in FIG. 11F, may be configured to simultaneously release two or more latches 712. The locking element may also be configured with branched segments to permit parallel release of latches.

In other embodiments, locking element 722 may comprise an electrically conductive material that melts upon the application of sufficient electrical current to permit the release of latch 712. In still other embodiments, the releasable retaining mechanism may comprise magnetic controlled locks or electropolymers embedded in latch 712 that may be controlled with application of current to wires embedded in tubular body 702 between latches 712 and the proximal end of guide tunnel 700.

Referring back to FIG. 11A, proximally, guide tunnel 700 may comprise one or more access ports. One or more of the ports 728, for example, may also be configured with a hemostatic seal to reduce blood loss during the procedure, and or with a reversible locking mechanism 730 to maintain the relative position between an inserted component and guide tunnel 700. Port 728 may be used for insertion and removal of the delivery catheter, for example. In some embodiments, one or more ports 732, 734 may be provided to obtain blood samples, for injection of radiographic or therapeutic agents, or for the attachment of a pressure transducer. Another port 736 may be provided for manipulation of locking element 722 which controls the release of latch structures 712.

In another embodiment, guide tunnel 700 further comprises an inner guide tunnel 750 that is removably insertable into passageway 703 of guide tunnel 700. In these and other embodiments comprising inner guide tunnel 750, port 728 that is configured to receive the delivery catheter will be located on the inner guide tunnel 750 while guide tunnel 700 will have a port 752 configured to receive the inner guide tunnel 750. Inner guide tunnel 750 further comprises an inner tubular body 754 with one or more openings 756 located at the distal end 758 of the inner tubular body 754. Opening 756 may be configured with flanking configurations or other configurations of radio-opaque markers that can be used to align opening 756 of inner guide tunnel 750 with the corresponding radio-opaque markers of latches 712. Opening 756 may comprise the same material as inner tubular body 754. In other embodiments, opening 756 is reinforced with a frame 806. In some embodiments, frame 806 may comprise a polymer of higher durometer than material comprising inner tubular body 754. In other embodiments, frame 806 may comprise a metal such as stainless steel, cobalt chromium, platinum-iridium, Nitinol or other nickel-titanium alloy. In further embodiments, frame 806 may be plated with an additional metal, including but not limited to gold. In some embodiments, frame 806 is plated with additional material to alter its radio-opacity. Inner guide tunnel 750 may also be configured with one or other proximal ports 734 previously mentioned.

In some embodiments, guide tunnel 700, inner guide tunnel 750 or the delivery catheter may include a position sensor system to detect the relative position of inner guide tunnel 750 and/or the delivery catheter. In one embodiment, the position sensor system comprises a series of electrical contact points along passageway 703 of guide tunnel 700 that can form an electrical circuit with one or more electrical contact points located on inner tubular body 754. Similarly, electrical contact points in the lumen of inner guide tunnel 750 can be used to detect the position of delivery catheters inserted therein. The position sensor system may be used as a substitute or in conjunction with radio-opaque markers to facilitate alignment of various components. Other types of position sensor system are also contemplated, including but not limited to optical and magnetic detection mechanisms.

In some embodiments, guide tunnel 700 with inner guide tunnel 750 may be used with delivery catheters comprising a single anchor, or delivery catheters with multiple anchors. In these embodiments, inner guide tunnel 750 may be used to simplify positioning of delivery catheters with respect to anchor openings 704 on guide catheter 700. Inner guide tunnel 750 may also be provided with one or more visual markings, detents, servo motor controlled positioning or other mechanisms to facilitate anchor delivery through anchor openings 704. In some embodiments, inner guide tunnel 750 may be configured, for example, to reorient end-firing anchor delivery catheters to deploy anchors through the side openings 705 of guide tunnel 700.

In some embodiments, guide tunnel 700 and inner guide tunnel 750 may be configured to restrict or limit any rotational movement between the two components. Such a feature may be useful where with more difficult target locations in the body that require considerable amounts of distance, angulation and torque to reach and may result in rotation and/or length misalignment. In one embodiment, depicted in FIGS. 12C to 12E, passageway 703 of distal section 706 is configured with a rail 800, groove or other alignment structure to resist rotational movement of inner guide tunnel 750. Rail 800 is attached at a distal end 804 and a proximal end (not shown) and permits inner guide tunnel 750 to longitudinally slide along between its two attachment points, where rail 800 passes through slots 802 or slits formed in the tubular body 754 of inner guide tunnel 750. In some embodiments, the rail has a width to thickness ratio of about 5:1 to about 20:1, preferably about 8:1 to about 16:1, and most preferably about 9:1 to about 14:1. In other embodiments, rail 800 is not attached proximally and permits inner guide tunnel 750 to be fully withdrawn from guide tunnel 700 and exchanged for a different inner guide tunnel 750. Rail 800 preferably comprises materials selected to reduce or minimize any friction or cohesion effects between the rail and the material comprising tubular body 754 of inner guide tunnel 750. In some embodiments, rail 800 may comprise a metal such as stainless steel or Nitinol. In other embodiments, rail 800 or other alignment configuration may comprise a lubricious coating such as PTFE to reduce movement resistance of inner guide tunnel 750. In still other embodiments, rail 800 may have a different cross-sectional shape from flat band configuration depicted in FIG. 12C, including but not limited to square, rectangle, circle, oval or other geometric shape.

Referring again to FIGS. 12A through 12H, a more detailed description of guide tunnel 700 is provided. FIG. 12A illustrates distal section 706 of guide tunnel 700. Distal section 706 is configured with a curvature configured to facilitate the placement of anchors in the subannular groove region. Seven anchor openings 706 are provided along the greater curvature 708 of distal section 706. In other embodiments, the number of anchor openings 706 may vary from about 2 or about 3, to about 30 or more. In preferred embodiments, anchor openings 706 may number from about 5 to about 20, while in most preferred embodiments, anchor openings 706 may number from about 7 to about 10. In some embodiments, anchor openings 706 may have a length of about 3 mm to about 20 mm, preferably about 5 mm to 10 mm and most preferably about 7 mm to about 8 mm. In some embodiments, anchor openings 706 may have a width of about 1 mm to about 10 mm, preferably about 2 mm to about 7 mm, and most preferably about 3 mm to about 5 mm.

The guide, mapping, delivery, and tunnel catheters provided in certain embodiments may be formed of any of a number of materials. Examples of suitable materials include polymers, such as polyether-block co-polyamide polymers, copolyester elastomers, thermoset polymers, polyolefins (e.g., polypropylene or polyethylene, including high-density polyethylene and low-density polyethylene), polytetrafluoroethylene, ethylene vinyl acetate, polyamides, polyimides, polyurethanes, polyvinyl chloride (PVC, fluoropolymers (e.g., fluorinated ethylene propylene, perfluoroalkoxy (PFA) polymer, polyvinylidenefluoride, etc.), polyetheretherketones (PEEKs), and silicones. Examples of polyamides that may be included in a catheter include Nylon 6 (e.g., ZYTEL® HTN high performance polyamides from DuPont™), Nylon 11 (e.g., RILSAN® B polyamides from Arkema Inc.), and Nylon 12 (e.g., GRILAMID® polyamides from EMS-Grivory, RILSAN® A polyamides from Arkema Inc., and VESTAMID® polyamides from Degussa Corp.). In some variations, the catheter may be formed of multiple polymers. For example, the catheter may be formed of a blend of different polymers, such as a blend of high-density polyethylene and low-density polyethylene. While the wall of the catheter may be formed of a single layer, some variations of tunnel catheters may include walls having multiple layers (e.g., two layers, three or more layers). Furthermore, some variations of the catheters may include at least two sections that are formed of different materials and/or that include different numbers of layers. Additionally, certain variations of tunnel catheters may include multiple (e.g., two, three) lumens. The lumens or walls may, for example, be lined and/or reinforced (e.g., with braiding or winding). The reinforcing structures, if any, may be metallic or comprise a non-metal or polymer having a higher durometer. Although some of the embodiments described above have a lumen having a length substantially similar to the length of the catheter body for the insertion of a guidewire, in other embodiments, a rapid-exchange type guidewire lumen may be provided.

### V. Tracking Assembly

While imaging techniques such as fluoroscopy or CT scanning may be used serially during an implantation procedure to confirm the positioning of the anchor delivery system, the increasing levels of radiation exposure poses a risk to both the patient and the physician. Furthermore, the contrast dye used during fluoroscopy or CT scanning may increase the risk of kidney failure in the patient. Although alternate imaging modalities are available, such as ultrasound or MRI, these modalities may be impractical for certain reasons, including low image resolution and interruption of the implantation procedure to perform imaging.

In some embodiments, an anchor delivery system may include a tracking assembly that may be used to identify the location of one or more components of the anchor delivery system. The tracking assembly may be used in lieu of or in conjunction with imaging systems to identify the location of the anchor delivery system. In one embodiment, the use of a tracking system may reduce the risk from ionizing radiation or contrast dye. In further embodiments, the tracking assembly may facilitate the manual positioning of the anchor delivery system by the physician or by remote control from an automatic or semi-automatic positioning system.

The tracking assembly may be configured to determine the location of an anchor delivery system component relative to one or more reference locations. The reference locations may be external reference locations and/or internal reference locations. An internal reference location may be provided by a tracking element positioned in a known location in the body on a catheter, or incorporated into an existing implant, such as a cardiac rhythm management device, or even another portion of the anchor delivery system, for example. In some embodiments, by systematically moving or sweeping the tracking assembly along various dimensions of a body structure, a model or map of the body structure may be generated. In these embodiments, the range of movement as limited by the body structure are detected and used to construct a model or map of the body structure. In further embodiments, the accuracy of the tracking assembly may be improved by moving the tracking assembly to multiple locations and calibrating or correlating the tracking data to a corresponding CT scan or other imaging modality. Thus, in some embodiments, a composite model or map is produced from the combination of the tracking data and a CT or MRI scan. Devices and methods for correlating tracking data to an imaging study are discussed in U.S. Pat. No. 6,301,496, which is hereby incorporated by reference in its entirety.

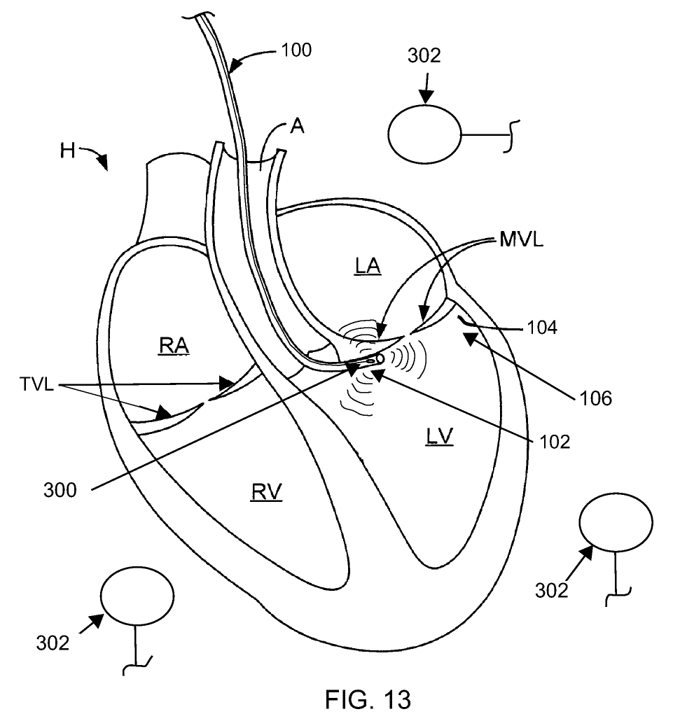

In one example, the tracking assembly comprises one or more trackable elements, such as a signal emitter, that are embedded in one or more components of the anchor delivery system. Referring to FIG. 13, in one embodiment, a signal emitter 300 incorporated into distal tip 102 of guide catheter 100. The location of signal emitter 300 may be determined by using the relative signal strength or other distance-based characteristic detected by the sensors 302 located internally and/or externally with respect to the patient. In one embodiment, at least one sensor 302 is located on guide catheter 100, from about 2 cm to about 20 cm, about 3 cm to about 10 cm, or a about 5 cm to about 10 cm proximal to signal emitter 300. In other embodiments, as depicted in FIG. 14, the sensor and signal emitter relationship may be switched, with sensors 302 embedded in one or more components of the anchor delivery system while one or more signal emitters 300 with known positions are provided internally and/or externally to the patient. Examples of magnet-based tracking assemblies and other tracking assemblies that do not require non-ionizing radiation are described in U.S. Pat. No. 5,713,946, U.S. Pat. No. 5,752,513, U.S. Pat. No. 6,690,963, and U.S. application Ser. No. 11/242,048, which are hereby incorporated by reference in their entirety.

In certain embodiments comprising a signal emitter, the signal may comprise any of a variety of signal types, including but not limited to magnetic signals, radiofrequency signals (FIG. 13), acoustic waves and electrical currents (FIG. 14). Likewise, the sensors may include antenna receivers configured to detect the radiofrequency signals, acoustic sensors, or electrodes configured to detect the electrical currents which may be used to generate impedance or voltage values. Examples of using electrical current from externally applied patch electrodes to identify the location of a catheter are described in U.S. Pat. No. 5,697,377, U.S. Pat. No. 5,983,126 and U.S. Pat. No. 7,263,397, which are hereby incorporated by reference in their entirety. In some embodiments utilizing electrical localization, correction of the measured may be provided to compensate for factors such as patient posture, respiratory phase, and cardiac contractile phase.

The type of tracking information provided by the tracking assembly can vary. In some embodiments, the tracking assembly is able to provide three-dimensional location data along X-, Y- and Z-axes with respect to one or more reference points. By providing continuous or sample-based tracking, directional, velocity or acceleration data relating to the movement of the tracking assembly can also be calculated from the location data, but in other embodiments, accelerometer sensors may be provided. In other embodiments, the tracking assembly may provide only two-dimensional location data along X- and Y-axes. In still other embodiments, tilting up-and-down (pitch), side-to-side (yaw) and/or turning left-and-right (roll) may be also be detected. These data types may be particularly useful when the tracking assembly is utilized with a remote control anchor delivery system, which is described in greater detail below.

In some embodiments, the tracking assembly optionally includes a mechanical sensor component. The mechanical sensor may comprise, for example, one or more strain gauges or piezoelectric material that can sense mechanical contact or pressure of the tracking assembly against a body structure. As the tracking assembly is moved or swept, information from the mechanical sensor component may be used to augment the positional data generated by the tracking assembly. The use of both mechanical sensor data and positional data may improve the accuracy of the anatomical mapping. In other embodiments, an imaging component such as an intravascular ultrasound assembly or an optical coherence tomography assembly, may be incorporated into the tracking assembly. Data from the imaging component may be used, for example, to determine the distance from the tracking assembly to the body structure surface, or to provide a two-dimensional or three-dimensional image of the adjacent tissue.

To account for variations in catheter position that may occur during the cardiac cycle, in some embodiments, data acquisition may be synchronized or organized to a particular reference point during the cardiac cycle, e.g. end-diastole or end-systole. Determination of the reference point may be performed based upon an external and/or intracardiac electrogram, or from the optional mechanical sensors that may be used to detect cardiac contractile activity. In further embodiments, variations relating to the inspiratory and/or expiratory phases of the respiratory cycle may also be taken into account. The respiratory cycle may be assessed, for example, by using intrathoracic pressure sensors, externally applied mechanical sensors and/or transthoracic impedance sensors.

### VI. Mapping Assembly

In other embodiments, the tracking assembly may optionally include a physiological sensor that can provide site-specific physiological information. For example, intrinsic electrical states or activity, or tissue impedance may be detected and associated with a particular location or structure on the model or map. This functional map of the body structure may be used as a guide for selecting anchor sites or sites for other treatments. A functional map of the body structure may also be used in some embodiments to distinguish infarcted myocardium from intact myocardium, or to distinguish myocardium from annular tissue. Although not bound by such a theory, it is believed that infarcted myocardium and annular tissue may be distinguished from intact myocardium by increased impedance and/or a reduction or lack of action potential conduction.

In one example, a functional map of the electrical conduction system of the heart may be generated from mapping electrodes used to assess the membrane potential or action potential at a location of the heart. Examples of mapping algorithms and components that may be used with various embodiments are described in U.S. Pat. No. 5,662,108, which is hereby incorporated by reference in its entirety, and U.S. Pat. No. 6,301,496, which was previously incorporated by reference. In one specific example, the myocardium along the subvalvular region of the mitral valve is checked for accessory or aberrant conduction pathways, such as Wolff-Parkinson-White Syndrome, prior to implantation of multiple anchors in that region. In some embodiments, electromapping of the cardiac tissue or heart chamber is performed during or after anchor deployment to check whether any alteration in membrane potential has been formed as a result of the anchors. Electromapping may also be performed after cinching an implant to assess its effect on conduction, if any, and may be performed before and/or after termination of the implant. One embodiment for diagnosing and treating accessory or bypass tracts in the subannular groove region is discussed below.

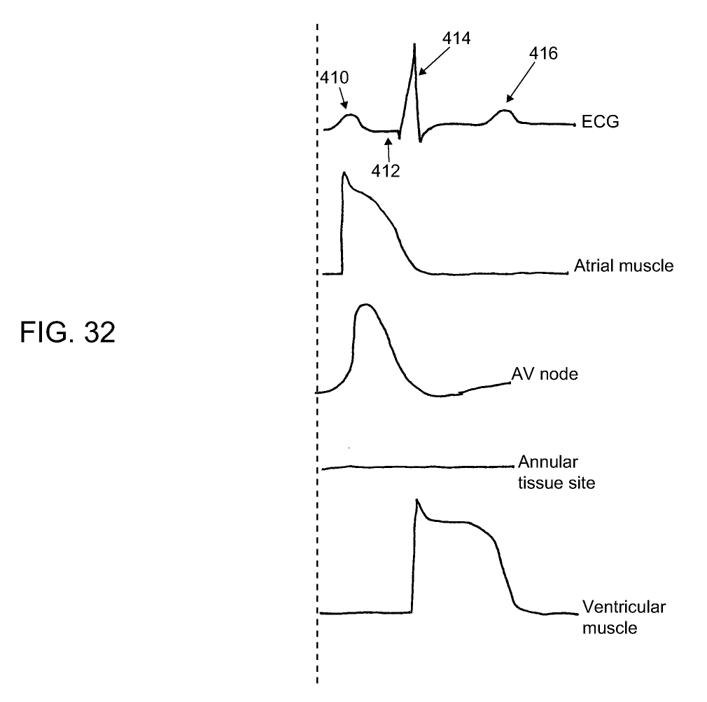

In patients with an intact cardiac conduction system, the depolarization of the myocardium of the heart chambers occurs in an organized fashion that optimizes the efficiency of the cardiac output of the heart. Typically, the depolarization starts spontaneously in the sino-atrial (SA) node located in the right atrium of the heart, and then spreads through the myocardium of the right atrium and then to the left atrium. Referring to FIG. 32, the depolarization of the atria forms the P wave 410 on an electrocardiogram (ECG) and normally lasts less than about 110 milliseconds. Next, the transmission from the atria to the ventricles normally occurs through the atrio-ventricular (AV) node, which introduces a delay in contraction ventricular contraction. This delay is represented by the PR segment 412 of an ECG, which is normally has a length of about 120 milliseconds to about 200 milliseconds. This delay results in earlier atrial contraction which moves atrial blood into the ventricles prior to ventricular contraction. This additional ventricular filling typically contributes about 10% to about 20% of the total filling of the ventricles, i.e. “atrial kick.” When atrio-ventricular contraction timing is disrupted, loss of atrial kick can decrease the stroke volume of the ventricles, resulting in a decrease in cardiac output. In some instances, increases in heart rate may compensate for reductions in stroke volume, but often times heart rate compensation results in further reductions in cardiac output as ventricular filling time is further reduced, resulting in even lower stroke volume.

From the AV node, electrical impulses are transmitted down the branches of the His bundle which results in nearly simultaneous contraction of the right and left ventricles, resulting the QRS complex 414 on the ECG, which has a normal duration of less than about 100 milliseconds. When normal ventricular depolarization is disrupted, QRS complexes with a larger duration are often formed. The repolarization of the ventricles may be seen on the ECG as the T wave 416. The SA node normally generates impulses at rates of about 60 to about 80 bpm. In patients where the SA node fail to generate spontaneous impulses, the AV node can take over spontaneous impulse generation at a rate of about 40 to about 60 beats per minute (bpm). When the AV node is also dysfunctional, the His bundle may take over at a lower rate of about 30 to about 40 bpm, or the ventricular myocardium may take over at a rate of about 20 to about 30 bpm.

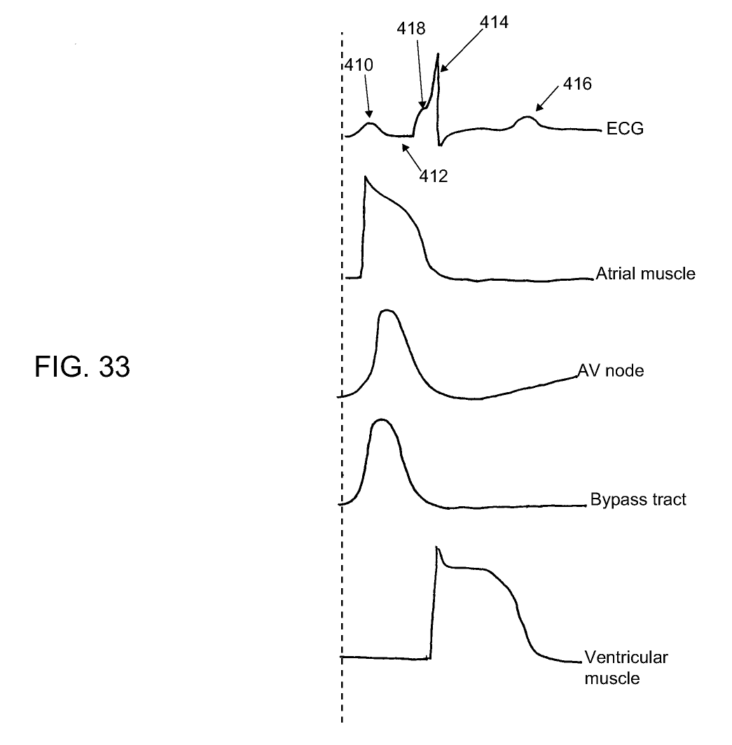

In patients with normal conduction systems, the annular tissue between the atria and the ventricles is electrically inert so that atrial depolarization does not propagate from the atria to the ventricles except through the AV node. In some persons, however, abnormal conduction pathways, known as an accessory or bypass tract, may exist between the atria and ventricles. In some cases, an electrical impulse may conduct back and forth between the bypass tract and AV node, resulting in a continuous circulating impulse than can cause ventricular heart rates greater than 250 bpm. Heart rates above 250 bpm fail to provide adequate forward blood flow and when sustained, may result in death. These tracts have a prevalence in the general population of about 0.1 to about 3.1 per 1000 persons. In some patients, the presence of a bypass tract may be evidenced by a delta wave 418 preceding the QRS complex 414 (FIG. 33). While these bypass tracts, which are called Wolf-Parkinson-White syndrome, may be treated with medications, mapping and ablating the bypass tracts are often curative.

While the ECGs of FIGS. 32 and 33 represent the composite electrical activity of the entire heart, mapping the myocardial surface of the heart permits the evaluation of the electrical activity of a defined location of the myocardium. FIGS. 32 and 33 further illustrate mapping of various sites of myocardium in a patient with a normal conduction system and one with Wolf-Parkinson-White syndrome, respectively. In addition to the manual tracings, however, electroanatomical maps may be depicted in a number of other ways, including but not limited to two-dimensional surface maps or three-dimensional models. Differences in membrane potential may be identified by dot density, grayscale, color or gradient line differences. Vector-based data, e.g. the general direction of action potential propagation by action potential timing differences between endocardial sites, may be depicted using by lines or arrows with a particular length, size and/or angular orientation. Examples of endocardial maps are depicted or discussed in U.S. Pat. No. 5,662,108, which was previously incorporated by reference, and U.S. Pat. No. 6,788,967, which is hereby incorporated by reference in its entirety.

In some embodiments, electromapping of a body structure may also be used as a method for confirming the location of a catheter. In patients where the electroanatomical map exhibits spatial variations, permitting catheter location to be determined by detecting membrane potentials along a length of a catheter and fitting the spatial pattern to a portion of the electroanatomical map to identify the catheter location. The electrodes may also be used to assess whether the guide tunnel or delivery catheter of the anchor delivery system is contacting the target tissue.

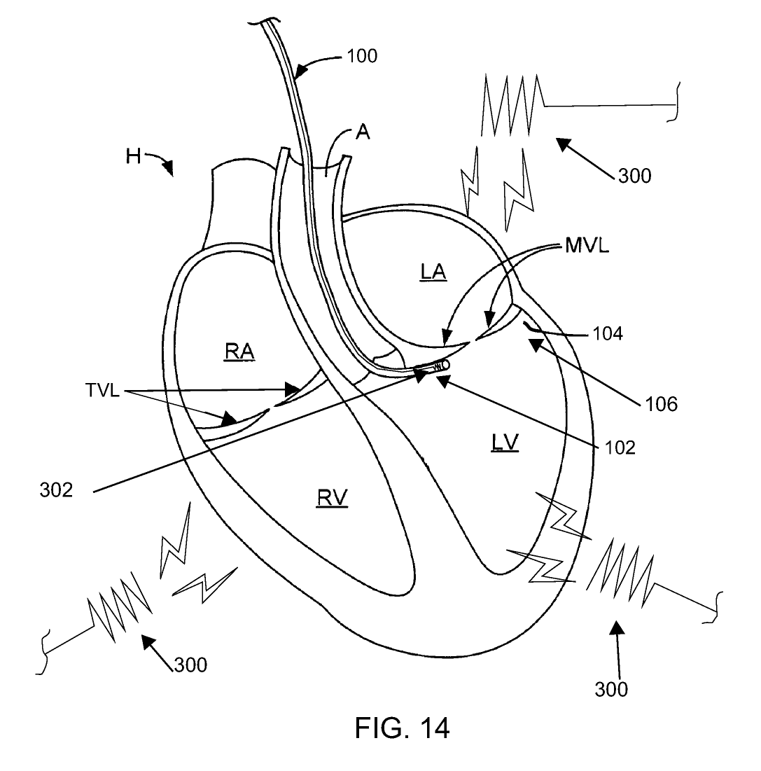

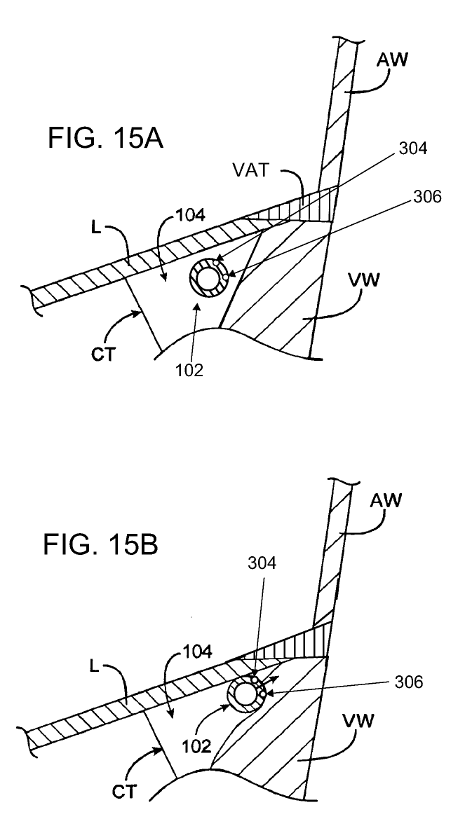

In one example, illustrated in FIGS. 15A and 15B, one or more pairs of electrodes 304, 306 located on distal portion 102 of a delivery device may be used to detect whether distal portion 102 is contacting the target tissue. By checking the membrane potential and/or impedance between electrodes 304 and 306, a lack of membrane potential or a low impedance may be used, for example, to determine whether distal portion 102 is merely in contact with the blood within the ventricle, as shown in FIG. 15A, or with the ventricular wall VW as shown in FIG. 15B. Although FIGS. 15A and 15B depict a distal end 102 of a delivery device using bipolar electrodes 304, 306, in other embodiments, a unipolar electrode may be used.

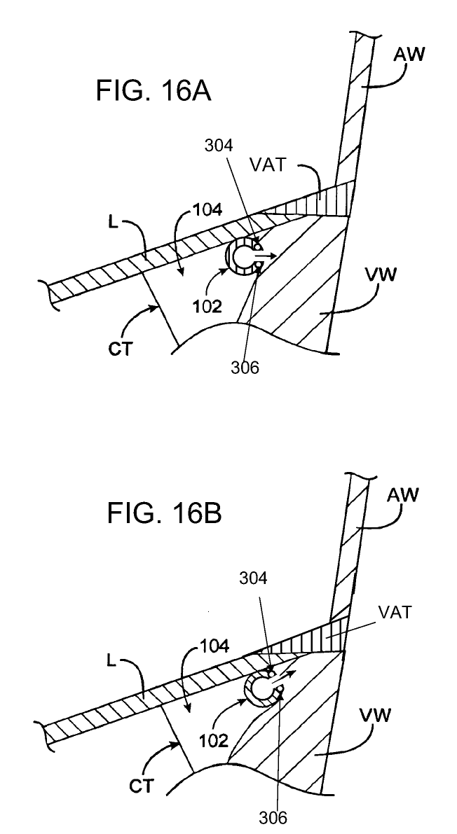

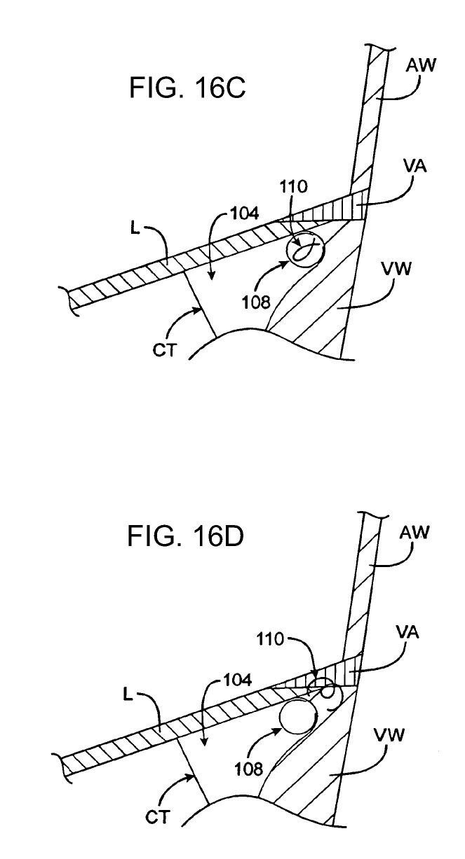

In another example, as illustrated in FIGS. 16A to 16D, distal portion 102 of the delivery device is positioned in a location under a valve leaflet L and adjacent a ventricular wall VW. The valve annular tissue VAT generally comprises an area of heart wall tissue at the junction of the ventricular wall VW and the atrial wall AW that is relatively fibrous. The term “annular tissue” as used herein shall include the valve annulus and the tissue adjacent or surrounding the valve annulus. Such tissue may exhibit a lower membrane potential or higher impedance than the tissue comprising the ventricular wall VW. In some instances, this physiological data may be used to reorient or angle the distal portion 102 of the delivery device, as depicted in FIG. 16B, to permit deploying an anchor 110 from delivery catheter 108 closer to the valve annular tissue VAT or away from the ventricular wall VW, as depicted in FIG. 16C and 16D. Although the mapping and detection procedures described herein are described in reference to the ventricles, mapping of the atria, pulmonary veins and coronary sinus may be performed.

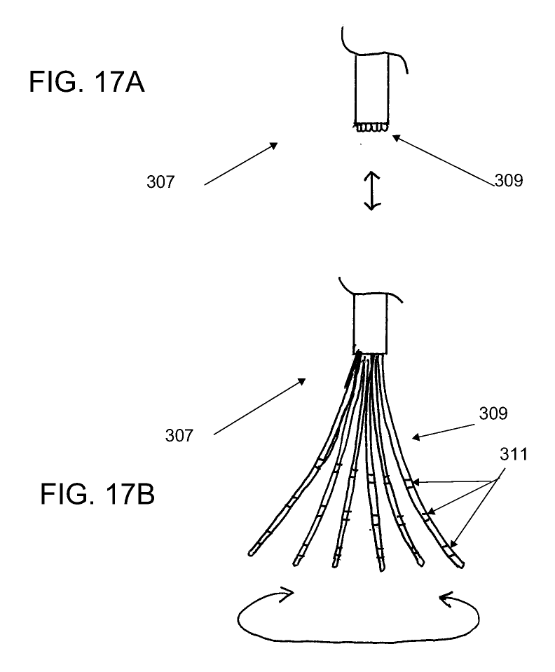

In some embodiments, the tracking assembly is provided on a separate tracking catheter or other dedicated component of the anchor delivery system. The tracking catheter is used to perform the initial modeling or mapping of the body structure prior to the implantation procedure. An increased number of sensors and/or an enlarged sensor structure may be provided on the tracking catheter to expedite the mapping process. FIGS. 17A to 19B, for example, depict various embodiments of tracking catheters with a plurality of sensors and/or tracking elements that may be used for anatomical or electroanatomical mapping of a body structure. FIG. 17A, for example, depicts a tracking catheter 307 comprising a plurality of elongate supports 309 with one or more tracking elements and/or sensors 311 located on each support 309. Elongate supports 309 are depicted with a single sensor 311 on their distal ends 313, but in other embodiments, sensors 311 may be located proximal to distal ends 313 or a plurality of sensors 311 may be located along the length of elongate supports 309. Elongate supports 309 may be straight, curved or have any other configuration and are optionally individually steerable or steerable as a group. In some embodiments, may curve or bend about 180 degrees backwards. This may facilitate mapping of the subannular groove region or subvalvular region of a ventricle. In other embodiments, the elongate supports 309 may have a general angulation anywhere from about 0 degrees to about 200 degrees, sometimes about 15 degrees to about 165 degrees, or about 30 degrees to about 90 degrees, or about 45 degrees to about 60 degrees. Elongate supports 309 need not have a uniform length, cross-sectional shape or cross-sectional area. In some embodiments, one or more elongate supports 309 are longitudinally movable and/or rotatable. One or more elongate supports 309 may be outwardly biased and/or sufficiently flexible so as to conform to the adjacent body structures. Elongate supports 309 may be arranged in a circular, elliptical, or other pattern. The pattern may be optimized, for example, for a particular heart chamber, and/or to avoid or increase contact with a particular region of the heart chamber. For example, in certain patients with wall segments exhibiting paradoxical motion during systole, one or more elongate supports 309 may be configured with a relatively greater outward position or bias to increase sensor 311 contact with that wall segment. The number of elongate supports 309 per tracking catheter 307 may vary, from about two to about sixteen or more, sometimes about three to about twelve or more, and other times about six to about eight or more.

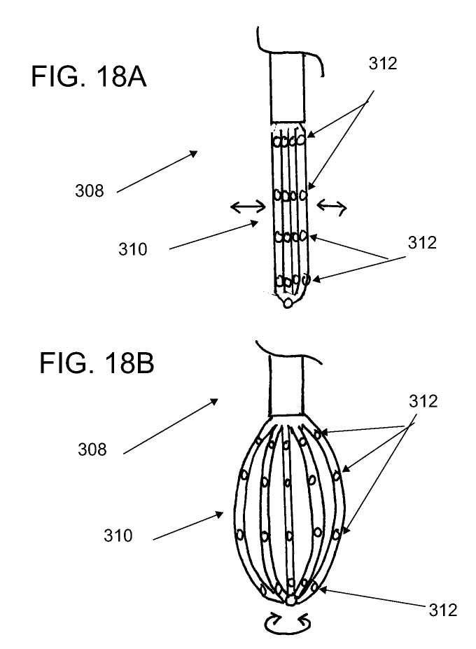

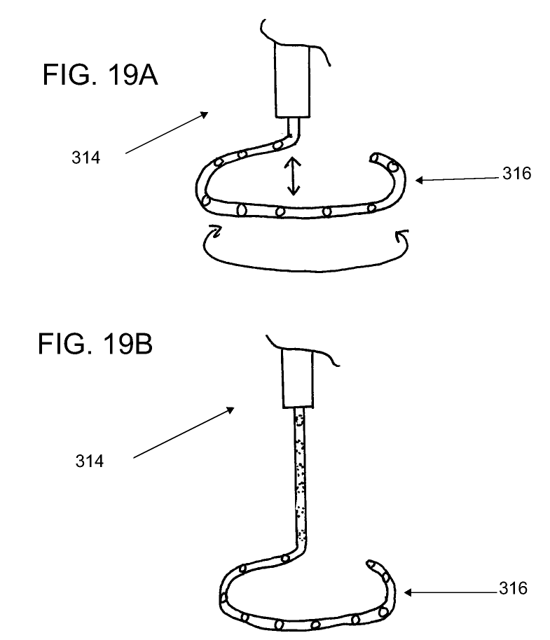

In some embodiments, as illustrated in FIG. 18A and 18B, the tracking catheter 308 comprises elongate supports 310 that are distally connected but may be manipulated to radially expand or contract. Tracking catheter 308 further comprises multiple sensors or tracking elements 312 on each elongate support 310. In still other embodiments, such as in FIG. 19A and 19B, the tracking catheter 314 may comprise one or more circumferentially configured elongate supports 316, in comparison to the longitudinally oriented supports 302 and 310 of FIGS. 17A to 18B. A tracking catheter 314 with a circumferentially configured member 316 may be better suited for identifying bypass tracts along the subannular groove region or subvalvular space, for example. Other examples of such tracking or mapping catheters are described, for example, in U.S. Pat. No. 5,156,151, and U.S. Pat. No. 5,297,549, which are hereby incorporated by reference in their entirety, and U.S. Pat. No. 6,301,496, which was previously incorporated by reference.

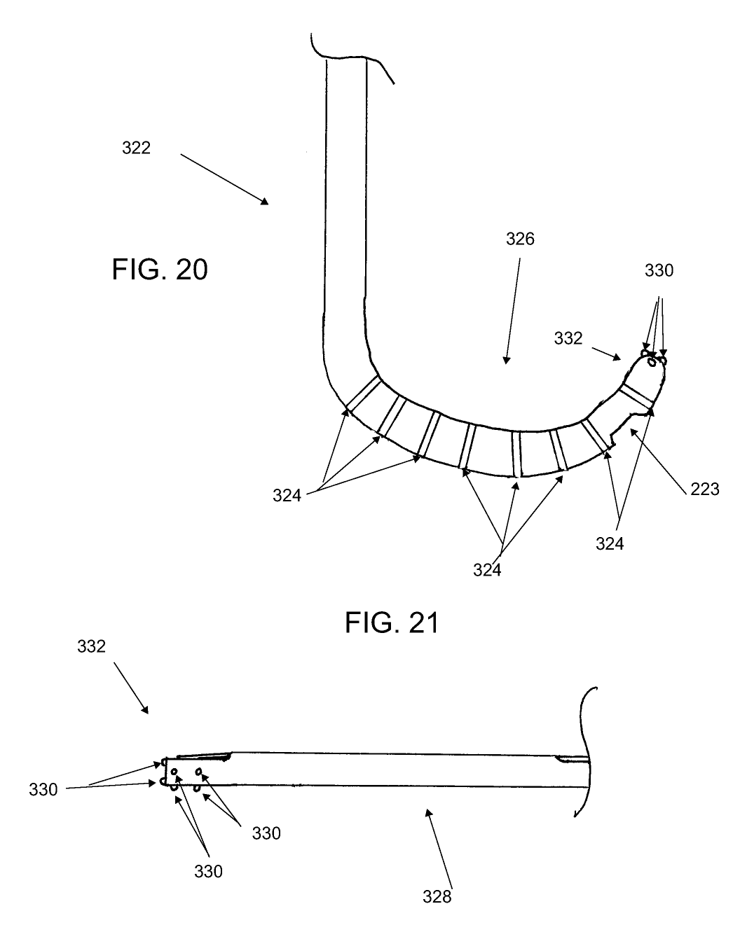

After the mapping procedure is complete, a separate delivery catheter with a sensor assembly of the same or similar modality may be used to deploy the anchors. The data obtained from this sensor assembly is compared to the model or map generated by the tracking catheter to assess catheter location. In other embodiments, however, the tracking assembly and delivery catheter may be integrated such that a separate tracking catheter is not required. For example, FIG. 20 depicts an embodiment of a guide tunnel 322 with a single aperture 323 and mapping electrodes 324 along a distal portion 326 of the tunnel 322 and mapping electrodes 330 about the distal tip 332, and FIG. 21 depicts an embodiment of a delivery catheter 328 with mapping electrodes 330 about the distal tip 332.

### VII. Remote Control

In some embodiments, the anchor delivery system may include one or more steerable catheters or components. The steerable catheter may have one or more uni-directional, bi-directional or multi-directional segments that may be manipulated using pullwires or electroactive polymers, for example. By controlling the orientation of the segment(s), the steerable catheter may be advanced a desired location, or to control the angle of anchor delivery with respect to the tissue surface, for example. In further embodiments, the steerable catheter may be coupled to a remote control system that can respond to instructions or commands from the user. The remote control system may also be configured to control the advancement or withdrawal of the catheter, the catheter rotation, and/or the bending of one or more steerable segments. In some embodiments, the control of the catheter or other component is performed by one or motors or actuators. The degrees of freedom controlled by the remote control system may vary depending on the device or procedure. Examples of such control systems are described, for example, in U.S. Pat. No. 6,726,675, U.S. Pat. No. 6,997,870 and U.S. Pat. No. 7,169,141, which are hereby incorporated by reference in their entirety. In some embodiments, the pullwires or conduction wires for electroactive polymers may directly interface with a mechanical or electrical-based controller of the remote control system. In other embodiments, manual controls, e.g. knobs, sliders and/or switches, are provided on the catheter. These manual controls may be manipulated by a person or by a mechanical controller of a remote control system configured to manipulate the manual controls.

The remote control system may also include sensors to detect resistance or structure contact during catheter manipulation and to cease or limit further attempts to guide the catheter to its desired location. Such sensors can act as a safety feature to reduce the risk of rupture or other trauma to adjacent body structures. Catheter movement by the motor control system can also include a feedback mechanism using the tracking assembly to further confirm that the intended catheter movement is occurring. In some embodiments, these sensors may be the same sensors as used by the tracking assembly to detect contact with body tissues or structures.

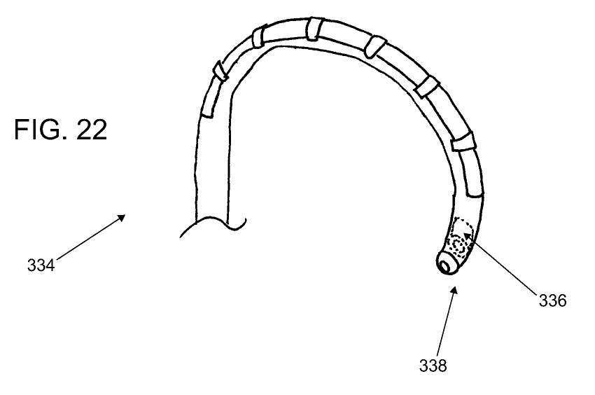

In other embodiments, the remote control system may comprise a magnetic guidance system. A magnetic guidance system utilizes external magnets to orient or move one or more magnets attached to a component of the anchor delivery system. The anchor delivery component may be a guidewire or a catheter, for example. FIG. 22 depicts one embodiment of a multi-window guide tunnel 334 with a magnet 336 embedded in its distal tip 338. Examples of magnetic guidance systems may be found in U.S. Pat. No. 6,524,303, herein incorporated by reference in its entirety, and U.S. application Ser. No. 11/242,048, which was previously incorporated by reference. As depicted in FIG. 22, in some embodiments, the magnet 336 (shown in ghost) embedded in guide tunnel 334 may comprise a tubular shape, such as a cylinder. Magnet 336 may comprise permeable or permanent magnetic materials such as barium/strontium carbonate ceramics, rolled steel, lanathoid elements, an iron-cobalt or a samarium-cobalt alloy, and neodymium-iron-boron (NIB). In some embodiments, a magnetically guided anchor delivery system may have certain advantages. For example, a manually manipulated catheter or a motor-controlled catheter may have an increased stiffness due to the presence of pullwires used to orient the catheter tip. In some instances, stiff catheters may have difficulty making tight bends in the body or may be difficult to navigate into small orifices. The tip of a magnetically guided catheter or guidewire, however, lacks pullwires and/or has a smaller diameter. Thus, in some embodiments, a magnetically guided catheter may have a greater bending range than a similar catheter with a pullwire. Likewise, in some embodiments, a magnetically guided catheter may easier to insert or guide into smaller or hard-to-reach orifices due to its smaller diameter. In other embodiments, a catheter with both magnetic and mechanical steering components may be used.

In some embodiments, the magnetic guidance system is used to orient the tip of the anchor delivery component, but in other embodiments, the magnet field may be used to push or pull the anchor delivery component along a pathway or portions thereof. In some embodiments, the magnetic field used to manipulate the magnet of the anchor delivery component may range in strength from about 0.15 T to about 3 T or more, sometimes about 0.25 T to about 2 T, and at other times about 0.5 T to about 1.5 T. Typically, two magnets are used to generate the magnetic field, but in some embodiments, a greater or lesser number of magnets may be used. In some embodiments, three or four magnets may be used.

### VIII. Energy Delivery and Cryotherapy

In some embodiments, one or more components of the anchor delivery system may include an energy delivery assembly. The energy delivery assembly may be a dedicated component of the anchor delivery system or may be incorporated in a guide catheter, mapping catheter, tunnel catheter delivery catheter or other component of the system. Energy delivery assemblies usable with various embodiments include but are not limited to thermal, radiofrequency (RF), ultrasound and laser-based assemblies. The energy delivery assembly may be used to ablate or tighten body tissues, facilitate penetration of therapeutic agent(s) into tissues, or to aid the reconfiguration of thermal-based shape memory anchors or guide elements, for example. In some embodiments, energy delivery may be provided at one or more target anchor sites, but in other embodiments, tissues or structures between the target anchors sites may be treated with energy delivery. In one specific example, it may be beneficial to perform ablation at the anchor target site in order to reduce any arrhythmogenic risk posed by anchor deployment into the myocardium or annular tissue, or to improve anchor penetration, for example. Energy delivery to any one site may occur before, during or after anchor deployment, tensioning of the tether, or termination of the tether. In some embodiments, energy delivery may be performed in a separate procedure before or after the anchor deployment procedure. The pre- or post-treatment energy delivery procedures may occur any time from about 1 hour to about 6 months or more from anchor deployment, sometimes about 24 hours to about 6 weeks, and other times about 3 days to about 3 weeks. The size and configuration of the treatment site(s) may vary. The dimensions of the energy delivery sites may range from about 0.25 cm² to about 10 cm², from about 0.5 cm² to about 5 cm², or about 1 cm² to about 2 cm² (as defined by the area in which about 50%, about 75%, about 90% or about 95% of the energy is delivered, or to which tissue damage occurs). The shapes of the energy delivery sites may be triangular, circular oval, square, rectangular, hourglass or any other shape. Shapes that are elongated may be generally oriented in a parallel or perpendicular fashion, or any orientation therebetween, with respect to the generally curvilinear arrangement of the deployed anchors.

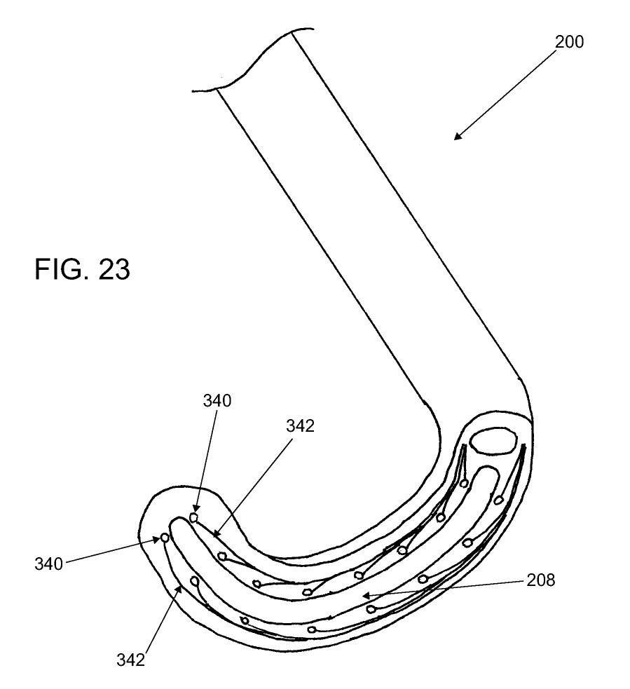

Embodiments using an RF-based energy delivery assembly, for example, may comprise a catheter with two or more electrodes located along a distal portion of the catheter and are attachable at its proximal portion to an energy source and an RF controller. In further embodiments, four, six, eight, ten, twelve, fourteen, sixteen, eighteen or twenty or more electrodes may be provided. In some embodiments, the electrodes are the same electrodes used to perform electrical-based tissue or structure mapping. The electrodes may comprises any of a variety of electrically conductive materials, including but not limited to copper, platinum, titanium, iridium, stainless steel, or combinations thereof, e.g. platinum-iridium. The electrodes may be recessed, raised or flush with the catheter surface. The electrodes may have any of a variety of shapes, including but not limited to band or ring-shaped electrodes, coil electrodes, point electrodes, or a combination thereof. The cross-sectional configuration of the electrode may be circular, elliptical, square, rectangular, triangular, polygonal, or any other shape. In embodiments of ring or coil shaped electrodes, the electrodes may have an average outer diameter of about 1 mm to about 4 mm or more, sometimes about 1.33 mm to about 3 mm or more, and other times about 1.66 m to about 2.33 mm or more. In embodiments with point-type electrodes, the electrodes may have an average diameter of about 0.25 mm to about 3 mm, sometimes about 0.5 mm to about 1.5 mm, and other times about 0.5 mm to about 1 mm. In embodiments with multiple electrodes, the electrodes may be arranged in any of a variety of configurations, including one or more longitudinally spaced arrangements to create curvilinear lesions using a length of the catheter. In other embodiments, the electrodes may be arranged into two or more groups or pairs. The spacing between the individual electrodes may vary from about 0.5 mm to about 4 mm or more, sometimes about 0.75 mm to about 3 mm or more, and other times about 1 mm to about 2 mm or more. Spacing between groups or pairs of electrodes may vary from about 2 mm to about 30 mm or more, sometimes about 3 mm to about 20 mm or more, and other times about 5 mm to about 10 mm or more. The wires used to conduct mapping information and/or current for energy delivery may comprise the same or different material as the electrode material. In some embodiments, the wires may have an average diameter of about 2/1000 mm to about 30/1000 mm or more, sometimes about 3/1000 mm to about 20/1000 mm or more, and other times about 5/1000 mm to about 10/1000 mm or more. The wires may be embedded or extruded with material used to form the catheter body or other elongate member. In some embodiments, one or more wires may be coated with an insulative or non-conductive material which is different from the material used for the catheter body. In some embodiments, the catheter body comprises a non-insulative or conductive material. FIG. 23, for example, is a schematic cut-away view illustrating one embodiment of an RF-energy delivery assembly comprising delivery device 200 of FIG. 8, further modified with eight pairs of spot-electrodes 340 and wires 342 arranged across delivery opening 208 and longitudinally spaced and positioned for energy delivery similar to the spacing and positioning for anchor deployment. Other electrode arrangements are discussed below.

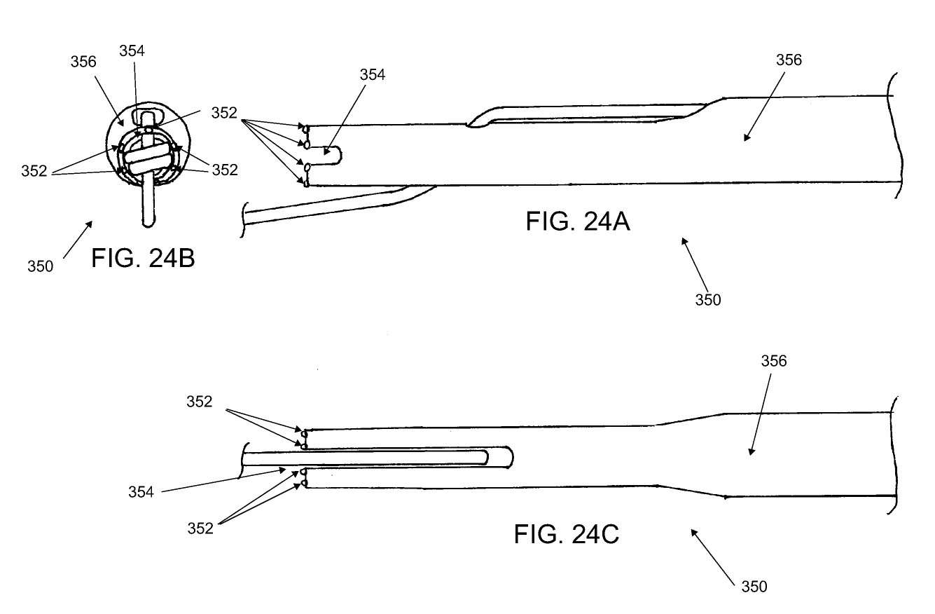

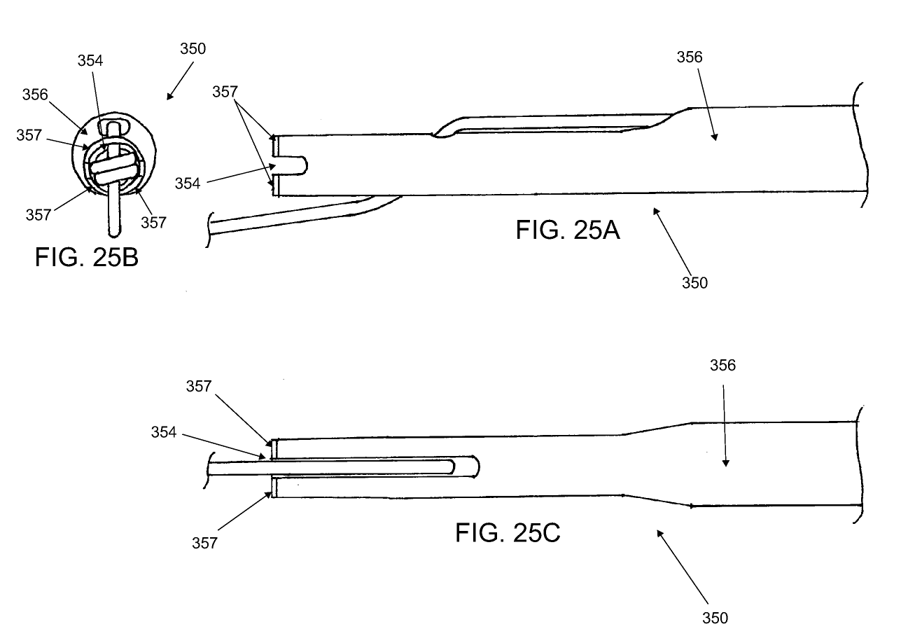

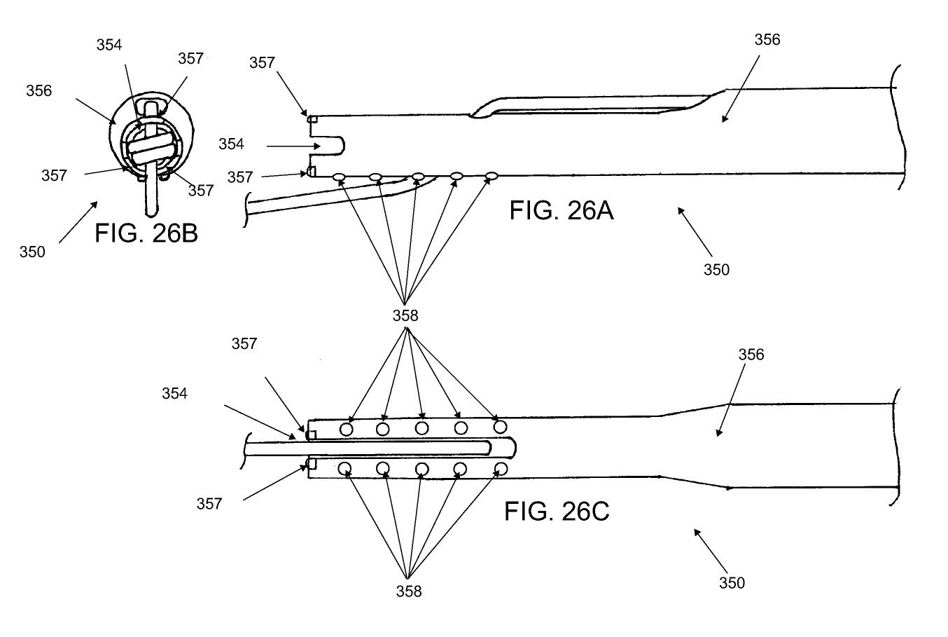

In some embodiments, the anchor delivery system comprises a delivery catheter with an energy delivery assembly and is usable with a tunnel catheter. Referring to FIGS. 24A to 24C, the delivery catheter 350 may comprise one or more electrodes 352 surrounding the anchor delivery aperture 354 of delivery catheter 350. Wires (not shown) contained within the body 356 of catheter 350, electrodes 352 are connected to a mapping controller, an energy delivery controller, or combination thereof. In some embodiments, the energy delivery controller may selectively map or ablate between any subset or pair of electrodes 352. In FIGS. 24A to 24C, electrodes 352 comprise spot electrodes, but in other embodiments as depicted in FIGS. 25A to 25C, arcuate electrodes 357 may be provided. Arcuate electrodes 357 may provide a greater surface area for energy delivery, as well as support for the anchor delivery aperture 354. Referring to FIGS. 26A to 26C, in other embodiments, additional electrodes 358 for mapping and/or ablation may be provided along a longitudinal length of delivery catheter 350. Additional electrodes 358 may be used, for example, in some instances where delivery catheter 350, in use, is tracked along a portion of the endocardium. Additional electrodes 358 may be used to determine the deployment angle of an anchor with respect to the endocardial surface. In the specific embodiment shown in FIG. 26C, additional electrodes 358 are provide along the tether slot 360 of delivery catheter 350.

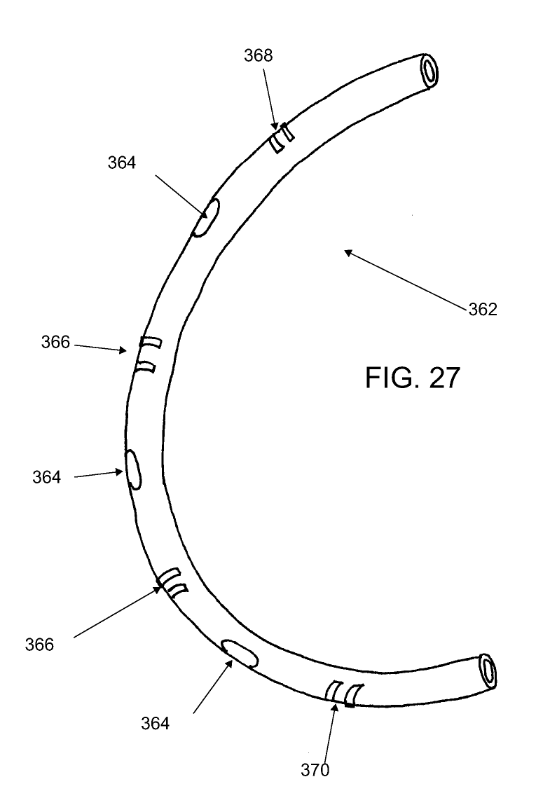

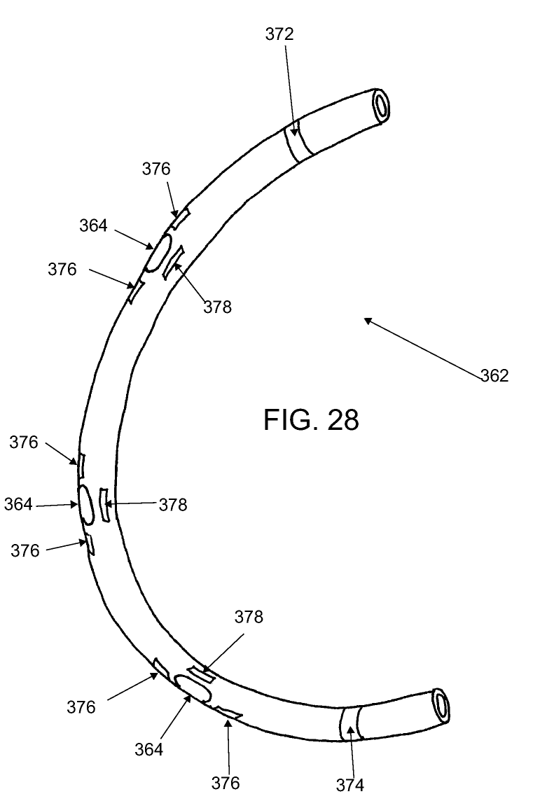

In other embodiments comprising delivery catheters with multiple anchor deployment apertures, the mapping and/or ablation electrodes may be configured in any of a variety of positions with respect to the apertures. In FIG. 27, for example, a catheter 362 with multiple apertures 364 comprises electrodes groups 366 located between the apertures 364, as well electrode groups 368 and 370 located proximal and distal to the apertures 364. In other embodiments, one or more sets of electrodes may be omitted. Although electrode groups 366, 368 and 370 as depicted are equally spaced from apertures 364, in other embodiments, one or more electrodes may be spaced a different distance from apertures 364. In still other embodiments, a single electrode or three or more electrodes may be provided in any one electrode group, and in some embodiments, two or more electrode groups may be provided proximal, between and/or distal to apertures 364. In some embodiments, performing ablation or energy delivery between delivered anchors may permit greater tensioning of the remodeled tissues by ablating or increasing the friability of the tissue adjacent the anchor site. Further, although the electrodes of electrode groups 366, 368 and 370 are depicted as partial arcs or rings in FIG. 27, any of a variety of electrode shapes may be used, including other shapes described herein. Also, electrode groups 366, 368 and 370 are shown as having a circumferential position similar to that of apertures 364, but in other embodiments, but in other embodiments, one or more groups may have an angular offset of about ±5 degrees to about ±180 degrees, sometimes about ±10 degrees to about ±135 degrees, and at other times about ±15 degrees to about ±90 degrees, and occasionally about ±30 degrees to about ±45 degrees. When used as mapping or tissue sensing electrodes, electrodes 366, 368, and 370 may be used to confirm tissue contact of the delivery catheter 362 prior to anchor deployment. FIG. 28 illustrates another embodiment of a catheter 362 with multiple apertures 364, and comprising proximal and distal band electrodes 372, 374, as well as longitudinal electrodes 376 and radial electrodes 378 adjacent the apertures 364.

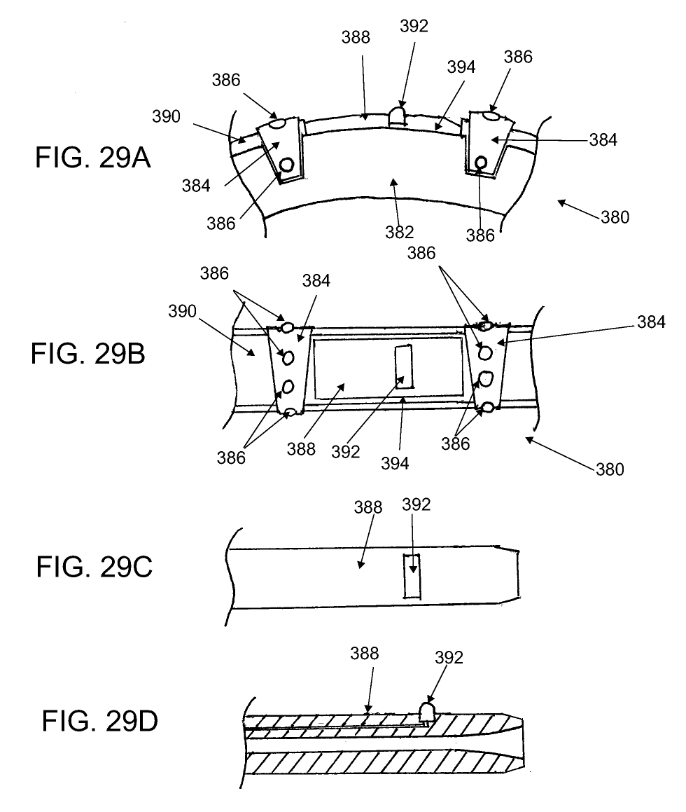

In other embodiments, the mapping electrodes and ablation electrodes may be located on separate components of the delivery system. In one embodiment, shown in FIGS. 29A to 29D, for example, a guide tunnel 380 comprising an outer catheter 382 with releasable retaining elements 384 and mapping electrodes 386 may be used with an ablation catheter 388 configured for insertion into the guide tunnel 380. Ablation catheter 388 may comprise one or more electrodes 392 through the openings 394 of guide tunnel 380. Toe reduce the risk of damage to guide tunnel 380, electrode 392 may be configured with a radial width that does not contact the inner lumen of guide tunnel 380. In other embodiments, guide tunnel 380 may be configured to resist RF damage and any ablation catheter, including those with full ring ablation electrodes, may be used. The embodiment depicted in FIGS. 29A and 29B comprises mapping electrodes 388 located on the releasable retaining elements 384, but in other embodiments, mapping electrodes 386 may also be located on the body of the outer catheter 382, and optionally the inner catheter 390 of the guide tunnel 380, and optionally on the ablation catheter 388 as well.

In some embodiments, the energy delivery assembly of the anchor delivery system includes a temperature sensor to detect the temperature of the treated tissue or body structure. The temperature sensor may be used as a feedback loop in the ablation controller to limit or stop energy delivery when certain temperature thresholds are reached. Such feedback loops may be used to limit the intended treatment zone to the desired target site. In some embodiments, the catheter comprising the energy delivery assembly may include one or more infusion or irrigation lumens. In some embodiments, one or more fluid may be passed through the lumen before, during and/or after an ablation treatment. In some instances, the fluid may be useful for controlling the degree of thermal effect surrounding the intended treatment site. The fluid may comprise chilled saline solution or chilled lactated Ringer's solution, for example. In other embodiments, unchilled fluids may be used. The volume or rate of fluid used may be varied or determined based upon the desired temperature control, the fluid status of the patient.

In some embodiments, an injection assembly and/or a cryotherapy assembly may be used in lieu of, or in combination with, the energy delivery assembly. An injection assembly may be used to inject one or more substances, including chemical or biological agents, into adjacent body tissue or structures may be provided. A cryotherapy assembly may utilize cooling substances, such as liquid nitrogen, to either destroy cells or to induce an immune response in the cells. In other embodiments, a cryotherapy assembly may be combined with a mapping assembly to assess the affect of cryotherapy on membrane potentials and conduction velocities of the myocardium. These cryotherapy-induced changes may be used to confirm conduction or other physiological abnormalities identified during mapping at normal temperatures, or to identify abnormalities not apparent at normal temperatures. Cryotherapy may also be used, for example, to cool or to control the thermal effects of other energy delivery components, or to cause temporary adhesion of the anchor delivery component to a treated site. Examples of cryotherapy catheters and assemblies may be found in U.S. Pat. No. 5,899,898 and U.S. Pat. No. 6,471,693, which are hereby incorporated by reference in their entirety.

### IX. Other Embodiments

Many of the features described herein may also be used a) in embodiments not involving the deployment of anchors, e.g. mapping and ablation of supraventricular or other arrhythmias, b) for deploying other types of implants or devices in the heart, and c) the deployment of cinching implants or anchors in other body systems such as the GI tract. Examples of these other embodiments are provided below.

A. Mapping and Ablation of Arrhythmias

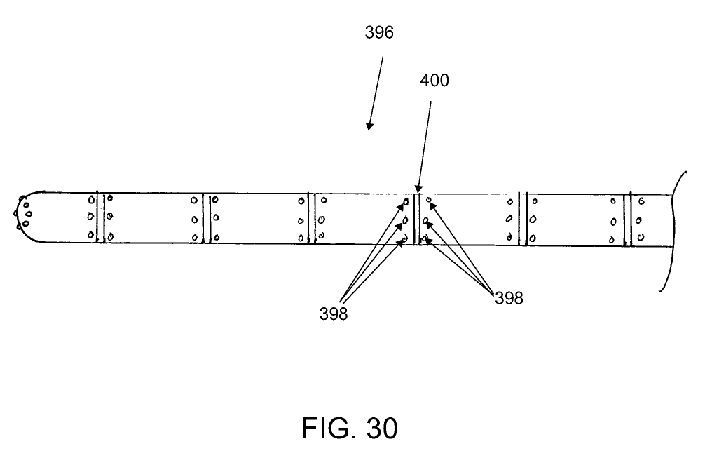

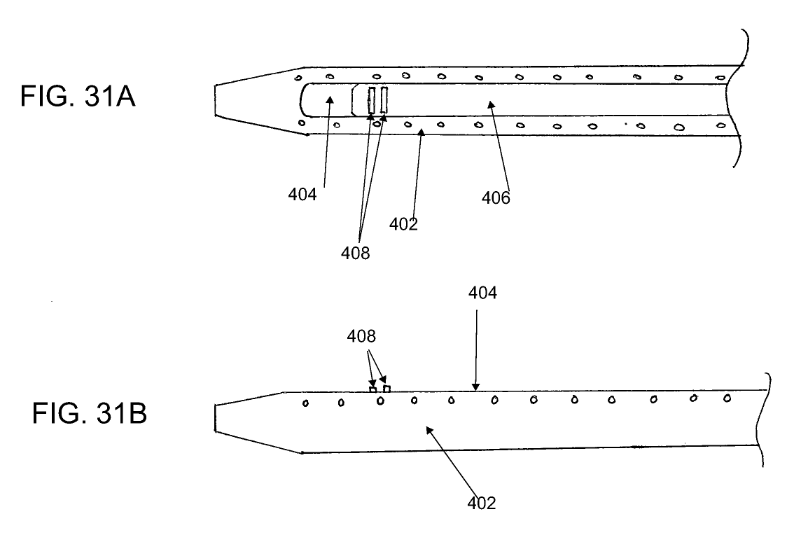

Referring to FIG. 30, in some embodiments, a mapping and ablation system is provided, comprising a mapping catheter 396 with a plurality of mapping electrodes 398 along its longitudinal length. To use the mapping and ablation system, access to the subannular groove region or other region of the heart may be achieved using any of variety of methods and access routes, including those described herein. The mapping catheter 396 may be positioned along the circumference of the subannular groove region and the activation data is obtained from the plurality of electrodes 398 to detect the accessory or bypass tract and/or common His bundle, for example. In some embodiments, the catheter 396 may be repositioned to confirm the detection of the bypass tract, or to further determine the exact location of the tract (where the spacing of the mapping electrodes is insufficient to detect activity along a generally continuous length of the subannular groove region). In the embodiment depicted in FIG. 30, the catheter 396 may further comprise a plurality of ablation electrodes 400 along the longitudinal length of the catheter. The ablation electrode(s) 400 at or closest to the detected bypass tract may be activated to ablate the tissue about the bypass tract and then the activation data may be reacquired from mapping electrodes 398 to confirm destruction of the tract. In this particular embodiment, no moving internal parts are necessarily manipulated to perform ablation at a selected location. In other embodiments, such as that depicted in FIGS. 31A and 31B, the mapping catheter 402 may comprise a through lumen in which an ablation catheter 406 may be inserted and positioned about the detected bypass tract. In comparison to the mapping guide tunnel 380 of FIGS. 29A and 29B, the lumen of mapping catheter 402 opens to a longitudinally oriented elongate opening 404 through which ablation may be performed anywhere along the length of the opening 404. The electrodes 408 may be configured to project about 0.5 mm to about 2 mm from the opening 404, but in other embodiments may be flushed or even recessed, depending on the type of energy delivery.

In still other embodiments, rather than a single opening, the catheter may comprise a plurality of longitudinal openings along a longitudinal length of the catheter, each opening associated with electrodes and/or other sensor configurations. In some examples, the electrodes and/or sensors may be used in a simultaneous or ordered fashion to assess the adjacent tissue without requiring movement of the catheter, and then a slidable treatment member within the catheter may be positioned at a selected opening based upon the electrode and/or sensor information to provide treatment and/or additional diagnostic testing through the selected opening. The treatment and/or additional diagnostic testing may include tissue ablation, implant or drug delivery, and/or biopsy. In some examples, the delivery instruments depicted in FIGS. 27, 28, and 29A to 29D may be used to provide treatment or to perform procedures other than anchor delivery. Thus, a mapping and ablation catheter may have any of a variety of electrode configurations as described in those embodiments, for example.

B. Anchor-Lead Devices

In some embodiments, an electrically conductive wire or lead may be coupled to an electrically conductive deployable anchor and used as a cardiac rhythm management lead. The lead may be configured as a sensing lead for a pacemaker, and/or as a pacing lead or a lead used for impedance measurement, for example. In some embodiments, the conductive lead may also function as a tether. In these embodiments, the conductive lead may be tensioned similarly to the tethers of other cinchable implants described herein and used with a non-cutting termination procedure to cinch the implant. Multiple anchors with multiple leads may be used with some embodiments. In other embodiments, the lead is separate from the tether or is used as a stand-alone cardiac rhythm management lead. The anchor-lead may be implanted using any of the variety of implantation procedures disclosed herein or incorporated by reference herein.

The anchor-lead may comprise a single metal or alloy, including but not limited to stainless steel, platinum-iridium, Ti—Nb—Zr alloy, Ni—Co—Cr alloy, Co—Cr—Mo alloy, titanium, and Ti-6Al-4V alloy. Alternatively, the anchor-lead may comprise two or more metals or alloys. For example, the anchor-lead may comprise one electrically conductive material to act as the pacing or sensing lead, while another material is used to provide the structural integrity and elasticity of the anchor. In a further example, a platinum-iridium material may be provided as an electrical conductor while a nickel-titanium material is provided for its shape-memory and superelastic properties. The anchor-lead may be coated with a polymer material to limit the surface area of activity sensing or the discharge of the electrical signal, including any of the polymer materials described previously. The insulative properties of the polymer or other material may vary depending upon the particular use. The portions of the anchor-lead configured to lie above the tissue surfaces after deployment may be coated with insulative material. These exposed portions may or may not include the tips of the arms and the portions adjacent to the lead coupling site, depending upon the particular anchor configuration and/or the tissue properties of the target site. In embodiments comprising two or more materials, electrically insulative materials may be provided may be provided between two or more materials to electrically isolate the materials. Furthermore, in anchor-leads comprising two or more materials, the configuration of the materials need not be symmetrical with respect to the lead coupling. For example, the electrically conductive material may be provided in only one of the arms of the anchor-lead. In other embodiments, the electrically conductive material may be provided in two or more arms, but the material in at least one of the arms is covered with an insulation material. In some embodiments, generally maintaining a balance between the structural or functional characteristics of the anchor-lead arms may be useful to provide an even deployment of the anchors.

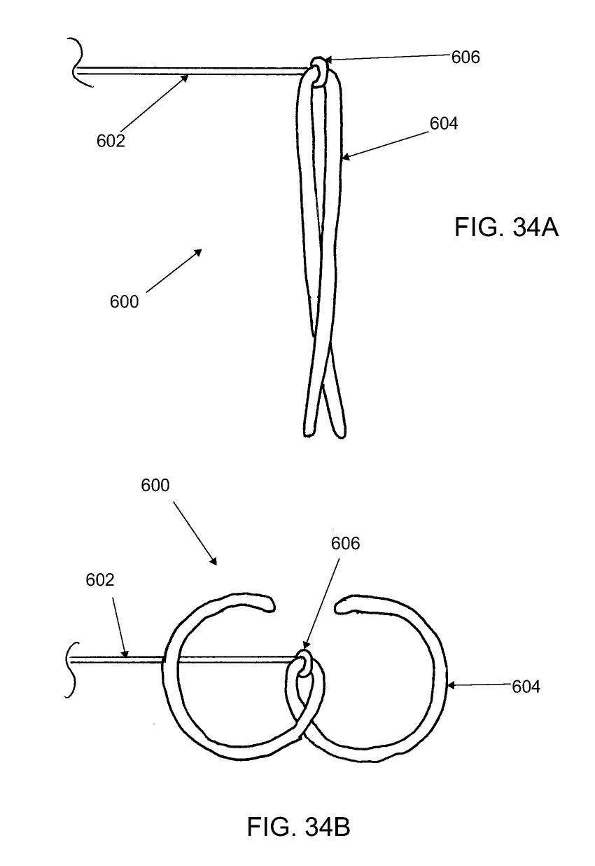

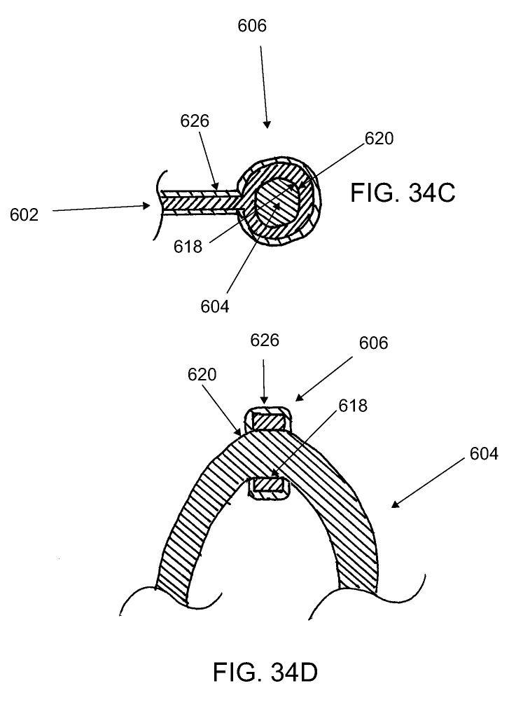

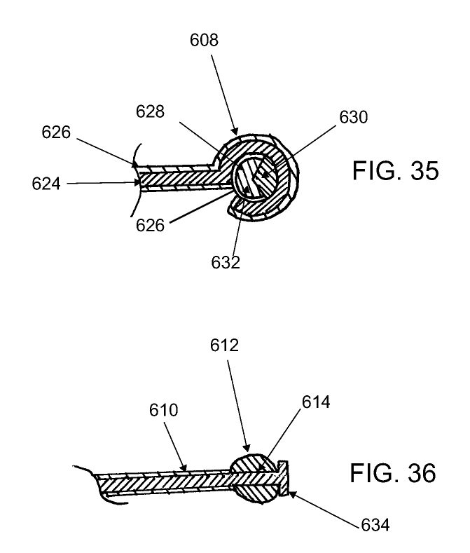

The coupling of the lead and the anchor may be configured to maintain electrical continuity between the two components. Referring to FIGS. 34A and 34B, one embodiment of an anchor-lead 600 may comprise an electrically conductive lead 602 and an electrically conductive anchor 604 which are secured together by a coupling 606. Coupling 606 may be a ring or loop-shaped lead coupling, but other coupling interfaces may be also used, including but not limited to C-shaped couplings 608 shown in FIG. 35, and leads 610 located in a through lumen 612 of the anchor body 614, depicted in FIG. 36, respectively. Referring to FIGS. 34C and 34D, the inner diameter 616 of the coupling ring 618 may have a tight tolerance with the outer diameter 620 of the anchor 604, but in other embodiments, a gap may be provided. In still other embodiments, the lead wire 602 and anchor 604 may be integrally formed, and a coupling is not required. In some embodiments, the tensioning of the lead wire, when used as a tether, maintains sufficient contact between coupling ring 618 and anchor 604. Coupling ring 618 and/or the lead wire 624 may be integrally formed as depicted in FIGS. 34C and 34D, or may be separately attached. Coupling ring 618 and/or lead wire 624 are optionally covered with a polymer or insulative material 626. Portions of anchor 604 may also be covered with insulated material 626.

The proximal end (not shown) of the lead wire 524 may be attached to any of a variety of pacemaker or cardiac rhythm management connectors, including but not limited to 3.2 mm type connectors, 5 mm type connectors, IS-1 type connectors, PSI Pacesetter type connectors, and 6L Cordis 6 mm bipolar connectors, for example. The lead connector may be pre-attached to the lead wire or may be attached to the lead wire after the lead wire has been sized and cut.

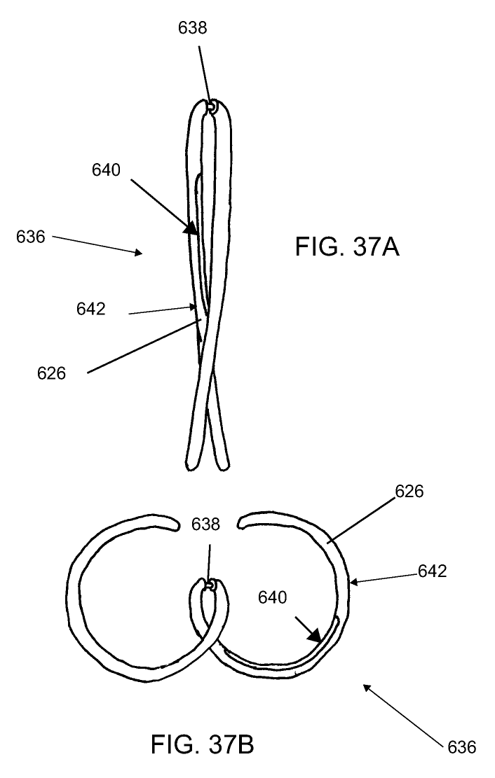

FIGS. 37A and 37B depict another embodiment of an anchor-lead 636, without a wire lead coupling attached. Anchor-lead 636 comprises an electrically conductive core having a coupling site 638 contiguous with an electrode site 640 located in an arm 642 of anchor-lead 636. The remaining portions of anchor-lead 622 are covered by insulative material 626. When deployed, anchor-lead 636 is electrically contacting the patient's body only at electrode site 640, while the other portions of the electrically conductive core remain generally isolated from the electrical activity of the body. The cross-sectional area and/or cross-sectional shape of the electrically conductive core need not remain constant along the longitudinal length of the core. For example, the surface area of the wire lead at electrode site 640 may be greater than coupling site 638, while coupling site 638 may have a higher volume to surface area ratio to resist breakage or fracture from tension, if any, exerted by the coupling or wire lead.

As discussed previously, FIGS. 35 and 36 depict alternate embodiments of a coupling. In FIG. 35, C-shaped coupling 608 contacts a portion of the anchor body 628 comprising two different metals. In this particular embodiment, a first metal 630 is provided as a conductor and a second metal 632 providing additional structural support. In other embodiments, but the functional or structural characteristics of the metals may differ in other ways. A portion of anchor body 628 may be covered with insulative material 626 to maintain insulation properties through a rotation range of C-shaped coupling 608. Lead wire 624 is also depicted with insulative material 626, but in other embodiments, a different material or no material may be used. In the other embodiment shown in FIG. 36, lead wire 610 comprises insulative material 626 and a flange or other blocking member 634. Blocking member 634 provides an interlocking fit between lead wire 510 and anchor body 514 that resists separation of the two components. While lumen 612 is depicted as a through lumen, in other embodiments, lumen 612 may be a closed lumen with a complementary configuration to blocking member 634.

C. Non-Anchor Implantable Components

In other embodiments, the components described herein may be used as-is or adapted with routine experimentation to deliver or deploy other cardiac components or perform other cardiac procedures. In one example, the multiple aperture catheters depicted in FIGS. 7, 9A and 9B, 11A to 11E and 22 may be used with a needle-tip catheter to inject one or more therapeutic agents into the myocardium or annular tissue. In other examples, the delivery systems may be adapted to implant cardiac rhythm management leads, pressure sensors and other elongate components.

D. Non-Cardiac Uses

In other embodiments, the components, including but not limited to the anchors and cinching implants, may be used for non-cardiac procedures. Any of a variety of tissue suspension procedures may be performed using the tethered anchors, both cinching or non-cinching versions. Bladder suspensions, face lifts, and breast augmentations may be performed, for example. Cinching implants may also be used to perform gastric reductions for the treatment of obesity, for example. The anchors may be inserted using an endoscope or laparoscope.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially.

What is claimed is:

1. A device for assessing tissue, comprising an elongate outer body, a plurality of longitudinally arranged sensor structures associated with a plurality of longitudinally arranged access regions of the elongate body, and wherein each sensor structure comprises a lead wire with a distal end coupled to a sensor structure and a proximal end located about a proximal portion of the elongate outer body.

2. The device of claim 1, wherein the sensor structure is an electrode structure.

3. The device of claim 1, wherein the plurality of longitudinally arranged access regions comprises a plurality of longitudinally arranged access openings.

4. The device of claim 1, further comprising a movable inner member within the elongate outer body and configured to be selectively positioned at each access region.

5. The device of claim 4, wherein the movable inner member comprises an ablation assembly.

6. The device of claim 4, wherein the movable inner member comprises a tissue injection assembly.

7. The device of claim 4, wherein the movable inner member comprises a sensor assembly.

8. The device of claim 4, wherein the movable inner member comprises an anchor delivery assembly.

9. A method for evaluating a patient for a cardiac abnormality, comprising:
positioning a catheter along a portion of an cardiac surface, wherein the catheter comprises a plurality of longitudinally arranged electrodes and at least two side openings;
assessing the physiological activity at a plurality of cardiac sites along the portion of the cardic surface without requiring repositioning of the catheter;
selecting an target site based upon the physiological activity of the plurality of cardiac sites;
positioning an active element at the target site; and
acting on the target site using the active element and at least one side opening of the catheter.

10. The method of claim 9, wherein the cardiac surface is an endocardial surface and wherein the plurality of cardiac sites are endocardial sites.

11. The method of claim 9, acting on the target site comprises ablating the target site using an active element that comprises an ablation element.

12. The method of claim 9, wherein assessing the physiological activity at the plurality of cardiac sites comprises assessing the physiological activity of at least two cardiac sites simultaneously.

13. The method of claim 10, wherein the portion of the endocardial surface comprises annular tissue associated with the mitral valve.

14. The method of claim 13, wherein the annular tissue is subvalvular annular tissue.

15. The method of claim 11, further comprising contacting the ablation element to the target site through a side opening.

16. The method of claim 15, wherein the ablation element is selected from a group consisting of radiofrequency ablation element, a cryoablation element and a high intensity focused ultrasound element.

17. The method of claim 9, wherein positioning the active element at the target site is performed without moving the catheter.

18. A tissue remodeling system for use in a patient, comprising:
an anchor delivery catheter comprising a through lumen and a first delivery aperture configured to releasably retain a biased anchor slidably coupled to a tether;
a tracking system configured for insertion into a body of a patient and comprising at least one electrode configured to acquire electrical information.

19. The tissue remodeling system as in claim 18, wherein the electrical information is tissue impedance information.

20. The tissue remodeling system as in claim 18, wherein the electrical information is membrane voltage information.

21. The tissue remodeling system as in claim 18, wherein at least a portion of the tracking system is embedded in a wall of the anchor delivery catheter.

22. The tissue remodeling system as in claim 21, wherein at least two surface electrodes are located about the first delivery aperture.

23. The tissue remodeling system as in claim 18, further comprising a tunnel catheter, wherein the tunnel catheter comprises a catheter lumen with at least one anchor aperture.

24. The tissue remodeling system as in claim 23, wherein at least a portion of the tracking system is embedded in a wall of the tunnel catheter.

25. The tissue remodeling system as in claim 20, wherein the tracking system further comprises an electrophysiology signal processor configured to receive a signal from the at least one electrode.

26. The tissue remodeling system as in claim 24, wherein the tunnel catheter comprises at least seven longitudinally spaced anchor apertures.

27. The tissue remodeling system as in claim 26, wherein the tracking system comprises at least eight electrodes.

28. The tissue remodeling system as in claim 27, wherein at least one electrode is located between each adjacent pair of longitudinally spaced anchor apertures of the tunnel catheter.

29. The tissue remodeling system as in claim 23, wherein the surface electrodes of the tracking system are at least double in number with respect to the number of anchor apertures of the tunnel catheter.

30. The tissue remodeling system as in claim 18, wherein the tracking system further comprises a catheter-embedded antenna assembly.

31. The tissue remodeling system as in claim 23, further comprising a magnetic navigation element.

32. The tissue remodeling system as in claim 31, wherein the magnetic navigation element is located at a distal portion of the delivery catheter.

33. The tissue remodeling system as in claim 31, further comprising a guidewire, wherein the magnetic navigation element is located at a distal portion of the guidewire.

34. The tissue remodeling system as in claim 18, further comprising an energy-delivery assembly.

35. The tissue remodeling system as in claim 34, wherein the energy-delivery assembly is integral with the anchor delivery catheter.

36. A method for securing an anchor to a body structure, comprising:
providing a first anchor;
positioning the first anchor at a first anchor deployment site;
assessing a physiologic property of the first anchor deployment site; and
deploying the first anchor at the first anchor deployment site.

37. The method for securing an anchor as in claim 36, further comprising changing the first anchor deployment site based upon the physiologic property.

38. The method for securing an anchor as in claim 37, further comprising reassessing the physiologic property of the first anchor deployment site after changing the first anchor deployment site.

39. The method for securing an anchor as in claim 36, wherein the physiologic property is an electrical property.

40. The method for securing an anchor as in claim 39, wherein the electrical property is a membrane voltage or a tissue impedance.

41. The method for securing an anchor as in claim 36, further comprising:
positioning a second anchor at a second anchor deployment site;
assessing a physiologic property of the second anchor deployment site; and
deploying the second anchor at the second anchor site.

42. The method for securing an anchor as in claim 41, further comprising retaining a tether coupled to the first anchor and the second anchor after deploying the first anchor and second anchor.

43. The method for securing an anchor as in claim 36, further comprising changing a tissue structure at the first anchor deployment site.

44. The method for securing an anchor as in claim 43, further comprising:
deploying the first anchor through a first opening of the catheter;
deploying the second anchor through a second opening of the catheter;
retaining the first coupling portion of the implant in the catheter, wherein the first coupling portion is located between two anchors secured to the body structure; and
releasing the first coupling portion of the implant from the catheter after securing the first anchor and the second anchor to body tissue.

45. The method for securing an anchor as in claim 36, wherein releasing the first coupling portion of the implant from the catheter comprises disengaging a wall section of the catheter.

46. The method for securing an anchor as in claim 36, further comprising positioning the catheter in a subvalvular space of a ventricle.

47. The method for securing an anchor as in claim 43, wherein changing the tissue structure at the first anchor deployment site comprises causing protein denaturation at the first anchor deployment site.

48. The method for securing an anchor as in claim 43, wherein changing the tissue structure at the first anchor deployment site comprises causing at least some tissue ablation at the first anchor deployment site.

49. The method for securing an anchor as in claim 41, further comprising cinching the first anchor and the second anchor closer together.

50. The method for securing an anchor as in claim 49, further comprising reassessing the physiologic properties of the first and second anchor deployment sites after cinching.

51. The method for securing an anchor as in claim 50, further comprising adjusting the cinching of the first anchor and the second anchor based upon reassessing the physiologic properties of the first and second anchor deployment sites.

52. The method for securing an anchor as in claim 50, further comprising securing the cinched first anchor and second anchor.

53. The method for securing an anchor as in claim 52, wherein securing the cinched first anchor and second anchor occurs after reassessing the physiologic properties of the first and second anchor deployment sites.

54. A method for assessing body tissue, comprising:
providing an image of a body structure constructed from localized body structure information;
positioning an anchor delivery system about the body structure, wherein the anchor delivery system comprises a sensor and an anchor coupled to a tether;
taking a localized information reading using the sensor of the anchor delivery system; and
comparing the localized information reading to the image of the body structure;
deploying the anchor at a target site of the body structure.

55. The method as in claim 54, further comprising:
repositioning the anchor delivery system based upon comparing the localized information reading to the image of the body structure.

56. The method as in claim 54, wherein the image of the body structure is a three-dimensional image.

57. The method as in claim 54, wherein the localized tissue information is electrical-based tissue information.

58. The method as in claim 54, wherein the localized tissue information comprises membrane potential data or impedance data.

59. The method as in claim 54, wherein the localized tissue information comprises tissue compliance data.

60. The method as in claim 59, wherein the tissue compliance data was generated using a catheter-based pressure sensor.

61. The method as in claim 54, further comprising determining an anchor delivery system location.

62. A method for treating body tissue, comprising:
accessing a plurality of cardiac target sites in a patient using a tubular body;
deploying a plurality of biased anchors at the plurality of cardiac target sites using the tubular body, wherein the plurality of biased anchors are coupled to a tether member;
delivering energy to at least one of the plurality of cardiac target sites using the tubular body in an amount sufficient to at least denature some protein at the at least one of the plurality of cardiac target sites; and
withdrawing the tubular body after deploying the plurality of biased anchors and after delivering energy to at least one of the plurality of cardiac target sites.
